SEQUENCE LISTING (7 sequences)

SEQ ID NO: 1 - protein, 337 aa, Thermoproteus tenax
MKIGVLTGGGDAPGLNIAVYTFVKLAERKHEVYAIYHGWRGLLNKEVKRVSSRDLLDFAFSGGTYIRTSRTNPFKDEERARLLESNVKELGLDVVVAIGGDDTLGAAGEAQRRGILDAVGIPKTIDNDVYGTDYTIGFDSAVNAAIEATESFKTTLISHERIGVVEVMGREAGWIALFTGLSTMADAVLIPERPASWDSVAKRVKEAYNERRWALVVVSEGIKEYGGPKDEYGHSRLGGVGNELAEYIERSTGIEARAVVLGHTIRGVPPTAFDRILAVRYATAAYEAVENGRYGVMVAYSNGDIAYVPIVDVVGKNRLVSGYWMRLYETYWPDLAG

SEQ ID NO: 2 - protein, 437 aa, Naegleria fowleri
MLSSSHLPTTIVTPKNVPTLGVLVGGGPAPGINGVIGAVTIEAINNGYRVLGFLEGFQNLILQDDSKIVELTIDSVSRIHFEGGSILKTSRANPTKKQEDLQKVVKQLQKFNVSLLVTIGGDDTAFSSMSVAKAANNEIHVVTLPKTIDNDLPLPYGIPTFGYETAREFGANVVRNLMTDASTASRYFIVVAMGRQAGHLALGIGKSAGSHLTLIPEEFLPTTDSTEPEVTFSRICDMIEASIIKRLYTSKKDHGVIVLAEGLLEYMSTDELKQAFGSSLKYDAHDHIMLAELDFGRLVRDEMRERMNRRGLKIAFTEKNLGYELRCAPPNAFDREYTRDLGNGAVRYLLNGGNGALITVQGVKMVPLSFDDLKDPRTGKTRTRQVDVSSEGFQVAKRYMIRLEKKDFEKEETLKGLAATAKCSVEDFIKQFKYLVQ

SEQ ID NO: 3 - protein, 420 aa, Methylococcus capsulatus
MAARNAFYAQSGGVTAVINASACGVLETARQYPDRIGTVYAGRNGIVGALTEDLIDTGQESAEAIAALRHTPSGAFGSCRYKLKGLEENRAQYERLIEVFRAHDIGYFFYNGGGDSADTCLKVSQLSEKLGYPLQAVHIPKTVDNDLPITDCCPGFGSVAKYIAVSVREASFDVRSMAATSTCIFVLEVMGRHAGWIAAAGGLASDERHELALVILFPEQVFDPERFLRAVDEKVRSHGYCSVVVSEGIRGADGRFVAESGSRDVFGHARLGGVAPVIADLIKERLGYKYHWAVADYLQRAARHIASRTDVEQAYAVGKAGVEMALKGLSAVMPAIVRTSDSPYRWEITAASLAEVANVEKKMPLEFISADGFGITEACRRYLRPLIEGEDYPPYAGGLPDYVTLCNVAVPKKLAASFSV

SEQ ID NO: 4 - protein, 341 aa, Amycolatopsis methanolica
MRVGVLTGGGDCPGLNAVIRAVVRKGIEAHGWEIVGFRSGWRGPLTGDSRPLGLDDVEEILIRGGTILGSSRTNPYKEEGGVEKIRAVLADQGVDALIAIGGEDTLGVAKKLTDDGIGVVGVPKTIDNDLAATDYTFGFDTAVHIATEAIDRLRTTAESHYRAMVVEVMGRHAGWIALHAGLAGGANVILVPERPFSVEQVVEWVERRFEKMYAPIIVVAEGAVPEGGAEVLRTGEKDAFGHVQLGGVGTWLADEIAERTGKESRAVVLGHTQRGGTPTAYDRVLATRFGLHAVDAVADGDFGTMVALRGTDIVRVKLAEATAELKTVPPERYEEAEVFFG

SEQ ID NO: 5 - DNA, 1014 bp, Artificial Sequence
  codon optimized coding region for WRI from oil palm
atgactcttatgaagaactctcctccttctacacctttgcctccaatttctcctagttcttctgcttctccaagttcttacgctccactttcttctccaaacatgatccctctcaacaagtgcaagaagtctaagcctaagcacaagaaagctaagaactctgatgagtcatctagaagaaggtcatctatctacaggggagtgactagacatagaggaactggaagatacgaggctcatctttgggataagcactggcaacatcctgttcaaaacaagaagggaaggcaggtttaccttggagcttttactgatgagcttgatgctgctagggctcatgatcttgctgctcttaaactttggggacctgagactatcctcaattttcctgttgagatgtacagggaagagtacaaagaaatgcagaccatgtctaaagaagaggttctcgcttctgtgagaagaagatctaacggattcgctagaggaacctctaagtatagaggtgtggctagacatcacaagaatggaagatgggaggctagactttctcaggatgttggatgcaagtacatctaccttggaacttacgctacccaagaagaggctgctcaggcttatgatttggctgctttggttcataagggacctaacatcgtgaccaactttgcttcttctgtgtacaagcacagactccaacctttcatgcagcttttggttaagcctgaaactgaacctgctcaagaggatcttggagttcttcaaatggaagctaccgagactatcgatcagactatgcctaactacgatctccctgagatctcttggaccttcgatatcgatcatgatcttggagcttaccctcttctcgatgttcctattgaggatgatcagcacgatatcctcaacgatcttaacttcgagggaaacatcgagcatctcttcgaagaattcgagacttttggaggaaacgagtctggatctgatggattttctgcttctaagggagcttga

SEQ ID NO: 6 - protein, 337 aa, Artificial Sequence
  Synthetic Construct
MTLMKNSPPSTPLPPISPSSSASPSSYAPLSSPNMIPLNKCKKSKPKHKKAKNSDESSRRRSSIYRGVTRHRGTGRYEAHLWDKHWQHPVQNKKGRQVYLGAFTDELDAARAHDLAALKLWGPETILNFPVEMYREEYKEMQTMSKEEVLASVRRRSNGFARGTSKYRGVARHHKNGRWEARLSQDVGCKYIYLGTYATQEEAAQAYDLAALVHKGPNIVTNFASSVYKHRLQPFMQLLVKPETEPAQEDLGVLQMEATETIDQTMPNYDLPEISWTFDIDHDLGAYPLLDVPIEDDQHDILNDLNFEGNIEHLFEEFETFGGNESGSDGFSASKGA

SEQ ID NO: 7 - DNA, 1254 bp, Artificial Sequence
  oleosin promoter
gctcgatcacacaaagaaaacgtcaaatgcatcatactgggcccattttgcagaccaagagaaagtgagagagagttgtcctctcgttatcaagtaacagtagaccaccactaaaccgcccaatagcttataatcaaaatagaaaggtctaataacagaaacaaatgaaaaagccttgttccatggactgcctacccgaattgattgattcgactagtttttcttcttctttgattaagacctccgtaagaaaaatggtactactaaagccactcgctaccaaaactaaaccattccagactgtaactggaccaatatttctaaactgtaaccagactctaaacatataaactaattaagaactataaccattaaccgtaaaaataaacttactacagtaaaaaattatactaatttcagctatgatggaatttcagctcttaagagttgtggaaatcaagtaaaattaaaatcataataatattcttcatccttatttttgtttcacatgcctgctgtccaatctgttattagcatttgaaagcctaaaattctatatacagtacaataaatctaattaattttcattactaataaaatgcttcatatatactcttgtatttataaatcatccgttatcgttactatacctttatacatcatcctacattcatacctaagctagcaaagcaaactactaaaagggtcgtcaacgacaagttatttgctagttggtgcatactacacacggctacggcaacattaagtaacacattaagaggtgttttcttaatgtagtatggtaattatatttatttcaaaacttggattagatataaaggtacagttagtgaaaatatttggttagcgggttgaattaaccggatataggagtcttatatacaactgtgaaaaaaggataaatacaaaaagggaagatgtttttccgacacacaagctaattaagtgcatcgagaggagagcaattgtaaaatgaatgttttgtttgtttttgtacggtggagagaagaacgaaaagatgatcaggtaaaaaatgaaacttggaaatcatgcaaagccacacctctcccttcaacacattcttacgtgtcgtcttctcttcactccatatctcctttttattaccaacaaatatatttcaatcccatttatatgtacgttctcttagacttatctctatatacccccttttaatttgtgtgctcttagcctttactctatagttttagatag

(19) United States
(12) Patent Application Publication
(10) Pub. No.: US 2017/0204426 A1
(43) Pub. Date: Jul. 20, 2017

(54) MODIFIED BRASSICA PLANTS WITH INCREASED SEED OIL CONTENT

(71) Applicant: BROOKHAVEN SCIENCE ASSOCIATES, LLC, Upton, NY (US)

(72) Inventor: Jorg SCHWENDER, Upton, NY (US)

(21) Appl. No.: 15/324,579

(22) PCT Filed: Jul. 7, 2015
(86) PCT No.: PCT/US15/39349

§ 371 Date: Jan. 6, 2017

Related U.S. Application Data
(60) Provisional application No. 62/021,992, filed on Jul. 8, 2014.

(51) Int. Cl. C12N 15/82; A23L 19/00; A23K 10/30; C12N 9/12

(52) U.S. Cl. CPC C12N 15/8247; C12N 9/1205; C12N 15/8218; C12Y 207/0109; A23L 19/00; A23K 10/30; A23V 2002/00

(57) ABSTRACT

Methods and means are provided to increase the seed oil content of Brassica plants by preventing feedback inhibition by phosphoenolpyruvate (PEP) of a pyrophosphate-dependent phosphofructokinase (PPi-PFK) present in cells of seeds or embryos of these plants, in various manners, including by providing feedback insensitive or less sensitive PPi-PFK.

This invention was made with Government support under contract number DE-AC02-98CH10886, awarded by the U.S. Department of Energy. The Government of the U.S.A. has certain rights in the invention.

The sequence listing that is contained in the file named “BCS 14-2004_ST25.txt” which is 24 kb (measured in operating system MS-Windows) and was created on Jun. 23, 2014, is filed herewith and incorporated herein by reference.

### FIELD OF THE INVENTION

The invention relates to the field of agronomy. More particularly, the invention provides methods and means to increase the seed oil content of Brassica plants by preventing feedback inhibition by phosphoenolpyruvate (PEP) of a pyrophosphate-dependent phosphofructokinase (PPi-PFK) present in cells of seeds or embryos of these plants, in various manners, including by providing feedback insensitive or less sensitive PPi-PFK, or by decreasing the steady-state level of PEP e.g. by increasing the level or activity of PEP carboxylase or PEP carboxykinase.

### BACKGROUND ART

Vegetable oils are increasingly important economically because they are widely used in human and animal diets and in many industrial applications, including as a renewable source to produce biofuel or biodiesel. The most widely used vegetable oils are derived from palm (world consumption 41.31 million tons in 2008) or soybean (41.28 million tons), followed by rapeseed oil (18.24), sunflower oil (9.91), peanut oil (4.82), cottonseed oil (4.99), palm kernel oil (4.85), coconut oil (3.48), and olive oil (2.84). Other significant triglyceride oils include corn oil, grape seed oil, hazelnut oil, linseed oil, rice bran oil, safflower oil and sesame oil.

Brassica napus is an important oilseed crop and an important bioenergy plant, with oil content ranging from 36 to 50% (on a dry-matter basis). In a key phase of seed development, also called “seed filling”, developing seeds can be considered metabolic sinks made up mostly of storage parenchyma cells that synthesize and store oils, protein or starch. As it is the case for many seed plant species, the principal storage organ within a B. napus seed is the embryo. The embryo is the filial generation which during growth and development within the seed has no symplastic linkage (cellular connection) to the mother plant. It autonomously takes up maternal carbon and nitrogen supplies (mostly sucrose, amino acids), biochemically transforms and distributes them into different storage compounds. A better understanding of the process of “carbon partitioning” between starch, lipids and proteins in developing seeds/embryos would allow increasing oil yield in a targeted manner.

The synthesis and deposition of storage compounds in oilseeds has been studied for a long time and is still subject of many recent research papers (for example: (Troncoso-Ponce et al., 2011: Comparative deep transcriptional profiling of four developing oilseeds. The Plant Journal 68: 1014-1027; Vigeolas et al., 2011: Nonsymbiotic hemoglobin-2 leads to an elevated energy state and to a combined increase in polyunsaturated fatty acids and total oil content when overexpressed in developing seeds of transgenic Arabidopsis plants. Plant Physiology 155: 1435-1444; Schwender and Hay, 2012 Mitochondrial metabolism in developing embryos of Brassica napus. Journal of Biological Chemistry 281: 34040-34047; Tang et al., 2012, Metabolic control analysis of developing oilseed rape (Brassica napus cv Westar) embryos shows that lipid assembly exerts significant control over oil accumulation. The New phytologist 196: 414-426). Past biochemical and genetic studies, mostly in the model species Arabidopsis, identified key steps and pathways in fatty acid and triacylglycerol biosynthesis, unraveled the genetic regulation of the expression of many components, and gave insight into how biosynthetic precursors (pyruvate, glycerol 3-phosphate) and energy cofactors arc provided in sufficient amounts to satisfy the high demands of fatty acid biosynthesis (Baud and Lepiniec, 2009, Role of WRINKLED1 in the transcriptional regulation of glycolytic and fatty acid biosynthetic genes in Arabidopsis. The Plant Journal 60: 933-947; Weselake et al., 2009, Increasing the flow of carbon into seed oil. Biotechnology Advances 27: 866-878).

Metabolic activity is generally controlled and regulated on different time scales and on different levels of cellular organization. Three main levels of regulation can be distinguished (Metallo and Vander Heiden, 2013: Understanding Metabolic Regulation and Its Influence on Cell Physiology. Molecular Cell 49: 388-398):
1) the regulation of gene expression, i.e. control of protein abundance via transcript level, translation, epigenetic effects etc. This might control expression of tissue specific enzyme isoforms.
2) Posttranslational modifications of enzymes that affect enzyme activity, such as phosphorylations.
3) Allosteric effects on enzymes by small molecules, which is also called “metabolic regulation”.
   While mechanisms of global transcriptional regulation have been the focal point of many studies, regulation at the biochemical level (metabolic regulation) is vastly under-explored.

Beyond the identification of essential parts of the metabolic network in developing oilseeds lies the question how flux through this network is controlled on a biochemical level. Flux through glycolysis and fatty acid synthesis might be strongly affected by allosteric activation or inhibition. In a broader sense, although some control mechanisms for plant glycolysis are known, it is unclear if those apply to developing oilseeds.

The involvement of phosphofructokinase in regulating glycolytic flux has been documented earlier. (Plaxton 1996: The organization and regulation of plant glycolysis, Annu. Rev. Plant Physiol. Plant Mol. Biol. 47:185-214). Transgenic plants comprising a heterologous phosphofructokinase gene have also been generated.

U.S. Pat. No. 7,012,171 describes a transgenic plant prepared by a method in which a plant cell is transformed with a chimeric gene comprising a promoter and a gene encoding a polypeptide which displays the activity of an enzyme which regulates the amount of a metabolic intermediate in glycolysis or in a pathway for the synthesis or degradation of starch, sucrose or reducing sugar from a glycolytic intermediate. In stored potatoes, an increased level of phosphofructokinase results in reduced accumulations of sugars in the tubers.

WO2006/127991 discloses methods of making monocotyledonous crop plants having higher oil levels in their seeds by increasing glycolytic flux through over-expression of nucleic acids encoding phosphofructokinase. More particularly, the document describes overexpression of the ATP-dependent phosphofructokinase from Lactobacillus delbreuckii subspecies bulgaricus in the seeds of monocots.

WO99/67392 is directed to the modification of metabolism in photosynthetic organisms. Photosynthetic organisms transformed with nucleic acid encoding an unregulated pyrophosphate:fructose 6-phosphate 1 phosphotransferase (PFP) protein capable of expressing a PFP protein which modifies metabolism without being regulated by the host organism, are described, as well as transgenic plants and seeds which yield modified carbohydrate, oil, fiber, sugar content as a result of alteration of glycolysis. More particularly, the document describes overexpression of a pyrophosphate-dependent phosphofructokinase gene from Giardia lamblia in tobacco plants.

The prior art does not describe which enzymes determine the carbon portioning between starch and lipid synthesis in developing embryos in oilseed rape, nor does it describe which molecules are responsible for metabolic regulation of these key enzymes. As described hereinafter, this problem has been solved, allowing the prevention or circumvention of feedback inhibition of pyrophosphate dependent phosphofructokinase by phosphoenolpyruvate in seeds and embryos in Brassica plants with the aim to increase lipid biosynthesis, as will become apparent from the different embodiments, examples and claims described herein.

### SUMMARY OF THE INVENTION

This invention involves preventing the feedback inhibition by phosphoenolpyruvate of a pyrophosphate-dependent phosphofructokinase (PPi-PFK) in cells of seeds or embyros of Brassica plants to increase the oil content in those seeds or embryos, e.g. by providing the plant with a PPi-PFK which is less sensitive to feedback inhibition by PEP than the endogenous PPi-PFK.

In a first embodiment, the invention provides a method to increase oil content in seeds or embryos of a Brassica plant, comprising the step of preventing feedback inhibition by phosphoenolpyruvate (PEP) of a pyrophosphate-dependent phosphofructokinase (PPi-PFK) present in cells of such seeds or embryos. The prevention of feedback inhibition may be achieved by providing the plant cell with a PPi-PFK variant which is less sensitive to feedback inhibition by PEP than a PPi-PFK endogenous to the plant. The variant may be encoded by a variant allele in the plant cell or by a transgene comprised within the plant cells.

When the PPi-PFK variant is a transgene, the coding region may be from an organism selected from the group of algae, bacteria, protozoa or archea, including Thermoproteus tenax, Naegleria fowleri, Methylococcus capsulatus or Amycolatopsis methanolica, wherein the PPi-PFK enzymes may comprise an amino acid sequence of any one of SEQ ID Nos: 1-4.

Thus, in one embodiment, the cells are provided with a DNA molecule comprising the following operably linked DNA fragments:
(a) a plant expressible promoter, preferably a seed-specific promoter;
(b) a DNA region encoding a PPi-PFK variant which is less sensitive to feedback inhibition by PEP than a PPi-PFK endogenous to the Brassica plant, preferably a DNA region encoding a PPi-PFK comprising an amino acid sequence selected from the amino acid sequences of SEQ ID Nos. 1, 2, 3 or 4, preferably a heterologous DNA region; or a DNA region encoding a protein 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity with an amino acid sequence selected from the amino acid sequence of SEQ ID Nos. 1,2, 3 or 4, and having PPi-PFK enzymatic activity; and optionally
(c) a transcription termination and/or polyadenylation region functional in plant cells.

The DNA region encoding the PPi-PFK may comprise a nucleotide sequence which is codon-optimized to codon usage in plants, preferably dicotyledonous plants, preferably Brassica plants.

In another embodiment, the invention provides a method as herein described comprising the further step of providing the plant cells with one or more further recombinant DNA molecules comprising the following operably linked DNA fragments:
(a) a plant expressible promoter, preferably a seed-specific promoter;
(b) a DNA region, preferably a heterologous DNA region encoding a polypeptide selected from the following group:
   (i) sucrose transporter capable of influencing sugar transport into the seed or embryo, such as AtSUC5;
   (ii) Na-pyruvate/sodium:proton antiporter;
   (iii) homeric acetyl-CoA carboxylase;
   (iv) glycerol-3-phosphate dehydrogenase;
   (v) transcription factor wrinkled 1, preferably comprising the amino acid sequence of SEQ ID No: 5 or a polypeptide having at least 90%, 91%, 92%, 93%, 94%, 95%, 96%, 97%, 98% or 99% sequence identity with amino sequence of SEQ ID No: 5;
   (vi) AtABCA9 transporter;
   (vii) Sn-2 acyltransferase
   (viii) lysophosphatidic acid acyl transferase
   (ix) glycerol-3-phosphate acyltransferase
   (x) diacylglycerol acyltransferase ; or
   (xi) oleosin; and optionally
(c) a transcription termination and/or polyadenylation region functional in plant cells.

In yet another embodiment, the invention provides a method as herein described comprising the further step of providing the plant cells with a further recombinant DNA molecule comprising the following operably linked DNA fragments:
(a) a plant expressible promoter, preferably a seed-specific promoter;
(b) a DNA region which encodes an inhibitory RNA capable of suppressing expression of sugar-dependent1 triacylglycerol lipase.

The invention also provides a method to increase oil content in seeds or embryos of a Brassica plant, wherein the prevention of feedback inhibition is achieved by reducing the steady state level of PEP in the plant cells, such as by increasing the level or activity of PEP carboxylase and/or PEPcarboxykinase including by introduction in the plant cells
of a recombinant DNA molecule comprising the following operably linked DNA fragments:
   i. a plant expressible promoter, preferably a seed specific promoter;
   ii. a DNA region encoding a PEP carboxylase or PEP carboxykinase; and optionally
   iii. a transcription termination and/or polyadenylation region functional in plant cells.

The Brassica plant may be Brassica napus, Brassica campestris (rapa), Brassica juncea or Brassica carinata.

The invention also provides Brassica plants, or seeds thereof, comprising in cells of its seeds or embryos, a pyrophosphate-dependent phosphofructokinase which is less sensitive to feedback inhibition by PEP than a PPi-PFK endogenous to a Brassica plant achieved according to the methods herein above described as well as oil derived from such plants.

Also provided by the invention are cells, tissues, oil storage tissue, embryos or seeds of a Brassica plant as herein described comprising a pyrophosphate-dependent phosphofructokinase which is less sensitive to the feedback inhibition by PEP than a PPi-PFK endogenous to the Brassica plant and/or comprising a recombinant PEP carboxylase or PEP carboxykinase and optionally further genetic modifications.

In a further embodiment of the invention, a chimeric DNA is provided comprising the following operably linked DNA fragments
a. a plant expressible promoter, preferably a seed-specific promoter;
b. a DNA region encoding a PPi-PFK which is less sensitive to feedback inhibition by PEP than a PPi-PFK endogenous to a Brassica plant, preferably a DNA region encoding a PPi-PFK comprising an amino acid sequence selected from the amino acid sequences of SEQ ID Nos. 1, 2, 3 or 4, preferably a heterologous DNA region; or a DNA region encoding a protein 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity with an amino acid sequence selected from the amino acid sequence of SEQ ID Nos. 1,2, 3 or 4, and having PPi-PFK enzymatic activity; and optionally
c. a transcription termination and/or polyadenylation region functional in plant cells.

The invention also relates to the use of a PPi-PFK enzyme which is less sensitive to feedback inhibition by PEP than a PPi-PFK endogenous to a Brassica plant to increase the oil content in seeds and or embryos of a Brassica plant.

Yet another embodiment of the invention is a method to isolate variants of PPi-PFK enzyme which are less sensitive to feedback inhibition by PEP than a PPi-PFK endogenous to a Brassica plant comprising the steps of
a. generating a multitude of variant PPi-PFK enzymes from a PEP feedback inhibition sensitive PPi-PFK from a Brassica plant;
b. identifying the enzymatic activity of each of the variant PPi-PFK enzymes in the presence of PEP;
c. isolating those enzyme variants which have a greater enzymatic activity in the presence of PEP than the enzymatic activity of the PEP feedback inhibition sensitive PPi-PFK.

The invention also provides a method to increase oil content in cells of a plant comprising the steps of
a. isolating a variant of PPi-PFK which is less sensitive to feedback inhibition by PEP according to the method herein above described; and
b. introducing the variant of PPi-PFK in a Brassica plant, preferably by transcription from a DNA construct encoding PPi-PFK.

The invention further relates to a method to isolate a plant cell or plant comprising a variant allele encoding a PPi-PFK variant enzyme which is less sensitive to feedback inhibition by PEP comprising the steps of
a. providing a population of plant cells or plants, each comprising a multitude of variant PPi-PFK;
b. identifying the enzymatic activity of each of the PPi-PFK enzymes in the presence of PEP;
c. isolating those plant cells or plants comprising enzyme variants which have a greater enzymatic activity in the presence of PEP than the enzymatic activity of the feedback inhibition sensitive PPi-PFK.

In another embodiment of the invention, a method is provided for producing food, feed, such as oil, meal, grain, starch, flour or protein or an industrial product, such as biofuel, fiber, industrial chemicals, a pharmaceutical or a nutraceutical comprising
a. obtaining the plant or a part thereof or a seed thereof as herein described; and
b. preparing the food, feed or industrial product from the plant or part thereof

### BRIEF DESCRIPTION OF THE DRAWINGS AND TABLES

FIG. 1: The experimental strategy for Example 1.

FIG. 2: The architecture and ultrastructure of the oilseed rape embryo cultured in vitro for ten days. (A) Embryo architecture in high- (CR3217) and low (CR2186) lipid entries. co: cotyledon, ra: radicle. (B) Storage parenchyma cells of the same two entries. Nuclei, vacuole, lipid bodies and cell wall were all visualizable by toluidine blue staining. The intensely stained structures arc starch grains. Inserts show individual starch grains surrounded by lipid bodies.

FIG. 3: Metabolic map of b144, with carbon net flux indicated by the thickness of the arrows. Numerical signs indicate significant correlations (p≦5%) with flux from plastidial fatty acid into lipids (vFASp), flux of Leu into protein (vLeuP) and flux of hexose phosphate into starch (vStout) (in that order). The numerical signs refer to the directionality of net flux, which may not be identical to the flux directionality definition in the metabolic network. Red arrows: effluxes into end product (lipid, protein and starch) accumulation. Grey arrows: statistically weakly determined flux values. The compartmentalized PEP/pyruvate node is highlighted in orange.

FIG. 4: Characterization of selected oilseed rape entries. (A) Principal component analysis based on LC/MS quantification of metabolites in cultured embryos. (B) Metabolite presence correlated with the accumulation of lipid, protein and starch; metabolites ordered according to their correlation coefficients.

FIG. 5: The proposed bottom-up control of glycolysis and starch synthesis in the developing seed. PEP and 3-PGA exert allosteric feedback control of upstream enzymes. Low levels of these intermediates drive carbon flow towards fatty acid/storage lipid synthesis, while an increase in their abundance shifts carbon flow toward starch synthesis. The red and blue colors associated with the arrows or text indicate, respectively, a significant positive or negative correlation with the accumulation of lipid. The levels of these two intermediates were highly inter-correlated.

FIG. 6: Selected significant correlations across the 9 genotypes (+ or − correlations) for values of net fluxes. All listed correlations are significant on a 2% level (p≦0.02) and the correlating fluxes are listed in order of declining p-values. Bold face: correlated fluxes are independent from the selected flux, i.e. not directly defined via stoichiometry of a connecting reaction.

FIG. 7: Quantitative relationship between measured enzyme capacity (Vmax) and related fluxes. The ratio Vmax/flux was derived for all seven genotypes for which enzymes were measured.

### DESCRIPTION OF DIFFERENT EMBODIMENTS OF THE INVENTION

The current invention is based on the combined and correlative analysis including determination of enzyme activities, metabolite concentrations and metabolic fluxes in in vitro cultivated embryos of nine genotypes of oilseedrape (Brassica napus) which are widely divergent in oil content. The correlative analysis demonstrated that increase in oil content across the different genotypes is caused in part by up-regulation of enzyme activity of plastidic pyruvate kinase, which is directly controlled by an oil seed specific transcription factor, Wrinkled1. In addition, at the metabolic level, a negative correlation of tissue levels of glycolytic intermediates to glycolytic flux is clearly evident, indicating dominance of a metabolic feedback in regulation of glycolysis. In particular, allosteric feedbacks by phosphoenol pyruvate (PEP) and 3-phosphoglycerate (3PGA) to upper glycolysis (phosphofructokinase) and to starch synthesis (ADP glucose pyrophosphorylase), respectively, appear to be the key mechanisms in regulating the partitioning of sugar supplies into oil and starch synthesis.

Accordingly, the invention provides a method for increasing the oil content in seeds and embryos of a Brassica plant by preventing feedback inhibition by phosphoenol-pyruvate of a pyrophosphate-dependent phosphofructokinase present in cells of the seeds and/or embryos.

In a first embodiment of the invention, the feedback inhibition is prevented by providing the Brassica plant cells with a PPi-PFK variant which is less sensitive to the feedback inhibition than a PPi-PFK endogeneous to the plant.

As used herein “pyrophosphate-dependent phosphofructokinase” or “PPi-PFK”, E.C. number 2.7.1.90, is an enzyme catalyzing the reversible interconversion of fructose-6phosphate and fructose 1,6-bisphosphate using inorganic pyrophosphate as the phosphoryl donor:

Diphosphate+D-fructose 6-phosphate⇄phosphate+D-fructose 1,6-bisphosphate

The systematic name of the enzyme is diphosphate:D-fructose-6-phosphate 1-phosphotransferase. Other names in common use include: 6-phosphofructokinase (pyrophosphate), inorganic pyrophosphate-dependent phosphofructokinase, inorganic pyrophosphate-phosphofructokinase, pyrophosphate-dependent phosphofructo-1-kinase, and pyrophosphate-fructose 6-phosphate 1-phosphotransferase or pyrophosphate-fructose 6-phosphate phosphotransferase.

Assays for measuring phosphofructokinase activity are well known in the art (see e.g. Reshetnikov et al. 2008, FEMS Microbiology Letters 288, 2, 202-210 or Alves et al. 1194, Journal of Bacteriology 176:6827-6835).

Genes encoding pyrophosphate dependent phosphofructokinases have been isolated and protein sequences thereof can be found in databases. The amino acid sequence of Arabidopsis thaliana PPi-PFK proteins can be found e.g. under Accession numbers Q8W4M5.1; Q9SYP2.1; F4JGR5.1; Q9C9K3.1; AEE28825.1; NP_172664.1; AEE30045.1; NP_173519.1; AEE82365.1; NP_192313.3; AEE82691.1; AEE35858.1; NP_680667.1 or NP_177781.1. Partial amino acid sequences (suitable for identification of full protein sequences and encoding genes) of Brassica PPi-PFK proteins can be found e.g. under Accession numbers ABV21226.1 or ABV21225.1. (herein incorporated by reference) Other amino acid sequences of PPi-PFKs from green plants are available.

One way to obtain PPi-PFK variant enzymes which are less sensitive to feedback inhibition by phosphoenolpyruvate is to isolate such variants starting from the amino acid sequences encoding PPi-PFKs, such as those mentioned or incorporated by reference herein, or their encoding nucleotide sequences, including those from plants.

To this end, a multitude of variant PPi-PFK enzymes or subunits thereof derived from a feedback inhibition sensitive PPi-PFK enzymes, preferably from a plant, such as a Brassica plant, can be generated using methods conventional in the art of protein engineering. For example, nucleotide sequences encoding PPi-PFKs may be subjected to PCR under error-prone conditions to create variants thereof. The variation may then even be enhanced using PCR to reassemble and shuffle these similar but not identical DNA sequences. Variant PPi-PFK enzymes may be expressed in host cells, such as E. coli or Saccharomyces cerevisae, Pichia pastoris, plant cells, Brassica plant cells etc. Next, the enzymatic activity of these variant PPi-PFKs is identified, in the absence and presence of phosphoenolpyruvate, as mentioned herein, and those enzyme variants (or their subunits) which have a greater enzymatic activity in the presence of phophoenolpuryvate than the enzymatic activity of the feedback inhibition sensitive PPi-PFK are isolated an optionally used to be introduced into Brassica plant cells.

Variant PPi-PFK enzymes (or monomeric polypeptides thereof) may also be generated in plant cells, encoded by variant alleles. To this end, a population of plant cells or plants comprising a multitude of variant PPi-PFK enzymes can be generated, e.g. through the use of mutagenesis. Again, the enzymatic activity of each of variant PPi-PFK enzymes in the presence of phosphoenolpyruvate is determined as herein described and those plant cells or plants comprising enzyme variants which have a greater enzymatic activity in the phosphoenolpyruvate than the enzymatic activity of the feedback inhibition sensitive PPFi-PFK (usually the endogenous PPi-PFK) arc identified. Plant cells may be used to regenerate plants comprising the variant alleles. These plants may be used in further crosses to combine the required variant alleles in the plant varieties of choice.

“Mutagenesis”, as used herein, refers to the process in which plant cells (e.g., a plurality of plants seeds or other parts, such as pollen, etc.) are subjected to a technique which induces mutations in the DNA of the cells, such as contact with a mutagenic agent, such as a chemical substance (such as ethylmethylsulfonate (EMS), ethylnitrosourea (ENU), etc.) or ionizing radiation (neutrons (such as in fast neutron mutagenesis, etc.), alpha rays, gamma rays (such as that supplied by a Cobalt 60 source), X-rays, UV-radiation, etc.), or a combination of two or more of these. Thus, the desired mutagenesis of one or more PPi-PFK encoding alleles may be accomplished by use of chemical means such as by contact of one or more plant tissues with ethylmethylsulfonate (EMS), ethylnitrosourea, etc., by the use of physical means such as x-ray, etc, or by gamma radiation, such as that supplied by a Cobalt 60 source. While mutations created by irradiation are often large deletions or other gross lesions such as translocations or complex rearrangements, mutations created by chemical mutagens are often more discrete lesions such as point mutations. For example, EMS alkylates guanine bases, which results in base mispairing: an alkylated guanine will pair with a thymine base, resulting primarily in G/C to A/T transitions. Following mutagenesis, plants can be regenerated from the treated cells using known techniques. For instance, the resulting seeds may be planted in accordance with conventional growing procedures and following self-pollination seed is formed on the plants. Alternatively, doubled haploid plantlets may be extracted to immediately form homozygous plants, for example as described by Coventry et al. (1988, Manual for Microspore Culture Technique for Brassica napus. Dep. Crop Sci. Techn. Bull. OAC Publication 0489. Univ. of Guelph, Guelph, Ontario, Canada). Additional seed that is formed as a result of such self-pollination in the present or a subsequent generation may be harvested and screened for the presence of mutant alleles. Several techniques are known to screen for specific mutant alleles, e.g., DeleteageneTM (Delete-a-gene; Li et al., 2001, Plant J 27: 235-242) uses polymerase chain reaction (PCR) assays to screen for deletion mutants generated by fast neutron mutagenesis, TILLING (targeted induced local lesions in genomes; McCallum et al., 2000, Nat Biotechnol 18:455-457) identifies EMS-induced point mutations, etc.

Another way to reduce feedback inhibition of PPi-PFK in Brassica plant cells by phosphocnolpyruvate is to use feedback insensitive PPi PFKs isolated from other organisms, such as algae, bacteria, archea or protozoa which possess that are not subject to allosteric inhibition by PEP.

Thus, a method is provided to increase oil content in seeds and/or embryos of a Brassica plant by providing the cells of such plant with a “phosphoenolpyruvate insensitive” PPi-PFK from algae, bacteria, archea or protozoa, such as the PPi-PFKs of Thermoproteus tenax, Naegleria fowleri, Methylococcus capsulatus or Amycolatopsis methanolica.

Characterization of these PPi-PFKs indicated that they are not inhibited by PEP (Reshetnikov et al. 2008, FEMS Microbiology Letters 288, 2, 202-210 or Alves et al. 1194, Journal of Bacteriology 176:6827-6835).

Suitable PPi-PFKs include the proteins/polypeptides with the amino acid sequence of SEQ ID Nos 1 to 4, and variants thereof. The nucleic acid molecules encoding these amino acid sequences for use in Brassica plants may be modified, for example, by codon optimization to facilitate expression in heterologous cells of Brassica cells, according to methods known in the art. This type of modification changes or alters the nucleotide sequence that encodes a protein of interest to use, throughout the sequence, codons that are more commonly used in the transgenic expression host cell. In addition, changes may be made to the nucleotide sequence that encodes the protein to adjust the relative concentration of A/T and G/C base pairs to ratios tha are more similar to those of the expression host. In addition, nucleotide sequences encoding the mutants of the invention may be further modified to encode other sequences such as those described above as being beneficial or desirable for inclusion in the modified proteins of the invention, e.g. sequences which target or direct the protein to a particular location or locations within the expression host cell, etc.

The term “variant” is intended to mean substantially similar sequences. Naturally occurring allelic variants can be identified with the use of well-known molecular biology techniques, as, for example, with polymerase chain reaction (PCR) and hybridization techniques as herein outlined. Variant (nucleotide) sequences also include synthetically derived (nucleotide) sequences, such as those generated, for example, by using site-directed mutagenesis. Generally, amino acid sequence variants of PPi-PFKS described herein will have at least 40%, 50%, 60%, to 70%, e.g., preferably 71%, 72%, 73%, 74%, 75%, 76%, 77%, 78%, to 79%, generally at least 80%, e.g., 81% to 84%, at least 85%, e.g., 86%, 87%, 88%, 89%, 90%, 91%, 92%, 93%, 94%, 95%, 96%, 97%, to 98% and 99% sequence identity to the amino acid sequences of the PPi-PFKs explicitly described herein, and will retain phosphofructokinase activity. Generally, nucleotide sequence variants have at least 40%, 50%, 60%, to 70%, e.g., preferably 71%, 72%, 73%, 74%, 75%, 76%, 77%, 78%, to 79%, generally at least 80%, e.g., 81% to 84%, at least 85%, e.g., 86%, 87%, 88%, 89%, 90%, 91%, 92%, 93%, 94%, 95%, 96%, 97%, to 98% and 99% sequence identity to the nucleotide sequences encoding the PPi-PFKs described herein, and the encoded products retain phosphofructokinase (either alone or in combination with other subunits).

Variants include, but are not limited to, deletions, additions, substitutions, insertions.

For the purpose of this invention, the “sequence identity” of two related nucleotide or amino acid sequences, expressed as a percentage, refers to the number of positions in the two optimally aligned sequences which have identical residues (x100) divided by the number of positions compared. A gap, i.e., a position in an alignment where a residue is present in one sequence but not in the other, is regarded as a position with non-identical residues. The “optimal alignment” of two sequences is found by aligning the two sequences over the entire length according to the Needleman and Wunsch global alignment algorithm (Needleman and Wunsch, 1970, J Mol Biol 48(3):443-53) in The European Molecular Biology Open Software Suite (EMBOSS, Rice et al., 2000, Trends in Genetics 16(6): 276-277; see e.g. http://www.ebi.ac.uk/emboss/align/index.html) using default settings (gap opening penalty=10 (for nucleotides)/10 (for proteins) and gap extension penalty=0.5 (for nucleotides)/0.5 (for proteins)). For nucleotides the default scoring matrix used is EDNAFULL and for proteins the default scoring matrix is EBLOSUM62.

Variant PPi-PFK encoding enzymes may be identified by hybridization. “Stringent hybridization conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridizes to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridizations (Northern blots using a probe of e.g. 100 nt) are for example those which include at least one wash in 0.2× SSC at 63° C. for 20 min, or equivalent conditions. “High stringency conditions” can be provided, for example, by hybridization at 65° C. in an aqueous solution containing 6× SSC (20× SSC contains 3.0 M NaCl, 0.3 M Na-citrate, pH 7.0), 5× Denhardt's (100× Denhardt's contains 2% Ficoll, 2% Polyvinyl pyrollidone, 2% Bovine Serum Albumin), 0.5% sodium dodecyl sulphate (SDS), and 20 μg/ml denaturated carrier DNA (single-stranded fish sperm DNA, with an average length of 120-3000 nucleotides) as non-specific competitor. Following hybridization, high stringency washing may be done in several steps, with a final wash (about 30 min) at the hybridization temperature in 0.2-0.1× SSC, 0.1% SDS. “Moderate stringency conditions” refers to conditions equivalent to hybridization in the above described solution but at about 60-62° C. Moderate stringency washing may be done at the hybridization temperature in 1× SSC, 0.1% SDS. “Low stringency” refers to conditions equivalent to hybridization in the above described solution at about 50-52° C. Low stringency washing may be done at the hybridization temperature in 2× SSC, 0.1% SDS. See also Sambrook et al. (1989) and Sambrook and Russell (2001).

Where the methods as described herein require providing the plant cell with a PPi-PFK variant which is less sensitive to feedback inhibition by PEP than the PPi-PFK encoding nucleic acid or acids endogenous to the plant cell, this may be conveniently achieved by expression of a recombinant transgene comprising the following operably linked DNA fragments:
a. a plant expressible promoter, preferably a seed-specific promoter;
b. a DNA region encoding a PPi-PFK variant which is less sensitive to said feedback inhibition than a PPi-PFK endogenous to said Brassica plant, such as a DNA region encoding a PPi-PFK comprising an amino acid sequence selected from the amino acid sequences of SEQ ID Nos. 1, 2, 3 or 4, preferably a heterologous DNA region; or a DNA region encoding a protein 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity with an amino acid sequence selected from the amino acid sequence of SEQ ID Nos. 1,2, 3 or 4, and having PPi-PFK enzymatic activity; and optionally
c. a transcription termination and/or polyadenylation region functional in plant cells.

As used herein, the term “plant-expressible promoter” means a DNA sequence which is capable of controlling (initiating) transcription in a plant cell. This includes any promoter of plant origin, but also any promoter of non-plant origin which is capable of directing transcription in a plant cell, i.e., certain promoters of viral or bacterial origin such as the CaMV35S (Harpster et al., 1988 Mol. Gen. Genet. 212, 182-190), the subterranean clover virus promoter No 4 or No 7 (WO9606932), or T-DNA gene promoters but also tissue-specific or organ-specific promoters including but not limited to seed-specific promoters (e.g., WO89/03887), organ-primordia specific promoters (An et al., 1996, The Plant Cell 8, 15-30), stem-specific promoters (Keller et al., 1988, EMBO J. 7, 3625-3633), leaf specific promoters (Hudspeth et al., 1989, Plant Mol Biol 12, 579-589), mesophyl-specific promoters (such as the light-inducible Rubisco promoters), root-specific promoters (Keller et al.,1989, Genes Devel. 3, 1639-1646), tuber-specific promoters (Keil et al., 1989, EMBO J. 8, 1323-1330), vascular tissue specific promoters (Peleman et al., 1989, Gene 84, 359-369), stamen-selective promoters (WO89/10396, WO 92/13956), dehiscence zone specific promoters (WO 97/13865) and the like. Other useful promoters include the nopaline synthase, mannopine synthase, and octopine synthase promoters, which are carried on tumor-inducing plasmids of Agrobacterium tumefaciens; the cauliflower mosaic virus (CaMV) 19S and 35S promoters; the enhanced CaMV 35S promoter; the Figwort Mosaic Virus 35S15 promoter; the light-inducible promoter from the small subunit of ribulose-1,5-bisphosphate carboxylase (ssRUBISCO); the EIF-4A promoter from tobacco (Mandelet al. (1995) Plant Mol. Biol. 29:995-1004); corn sucrose synthetase; corn alcohol dehydrogenase I; corn light harvesting complex; corn heat shock protein; the chitinase promoter from Arabidopsis; the LTP (Lipid Transfer Protein) promoters; petunia 20 chalcone isomerase; bean glycine rich protein 1; potato patatin; the ubiquitin promoter; and the actin promoter.

Seed specific promoters suitable according to the invention include but are not limited to: phaseolin, napin, 2S2 promoters the oilseed rape napin promoter (U.S. Pat. No. 5,608,152), the Vicia faba USP promoter (Baeumlein et al., Mol Gen Genet, 1991 , 225 (3):459-67), the Arabidopsis oleosin promoter (WO 98/45461), the Phaseolus vulgaris phaseolin promoter (U.S. Pat. No. 5,504,200), the Brassica Bce4 promoter (WO 91/13980) or the legumine B4 promoter (LeB4; Baeumlein et al., 1992, Plant Journal, 2 (2):233-9), the promoter sequences described in WO2009/073738; promoters from Brassica napus for seed specific gene expression as described in WO2009/077478; the plant seed specific promoters described in US2007/0022502; the plant seed specific promoters described in WO03/014347; the seed specific promoter described in WO2009/125826; the promoters of the omega_3 fatty acid desaturase family described in WO2006/005807 and promoters which bring about the seed-specific expression in monocotyledonous plants such as maize, barley, wheat, rye, rice and the like. Suitable noteworthy promoters are the barley Ipt2 or Ipt1 gene promoter (WO 95/15389 and WO 95/23230) or the promoters from the barley hordein gene, the rice glutelin gene, the rice oryzin gene, the rice prolamine gene, the wheat gliadine gene, the wheat glutelin gene, the maize zeine gene, the oat glutelin gene, the sorghum kasirin gene or the rye secalin gene, which are described in WO 99/16890. Also suitable promoters are those described in WO 2010/00708 or in WO 2010/057620 or WO2010/060609.

Other elements which enhance, control or optimize transcription and/or translation of the recombinant enzyme within the transgenic host include but are not limited to: various enhancer elements, e.g. various cis-acting elements within the regulatory regions of the DNA, trans-acting factors that include transcription factors, etc. One of more of these may also be included in the nucleic acid that contains the recombinant gene that is to be expressed in the host.

The recombinant DNA molecules as herein described optionally comprise a DNA region involved in transcription termination and polyadenylation. A variety of DNA region involved in transcription termination and polyadenylation functional in plants are known in the art and those skilled in the art will be aware of terminator and polyadenylation sequences that may be suitable in performing the methods herein described. The polyadenylation region may be derived from a natural gene, from a variety of other plant genes, from T-DNA genes or even from plant viral genomes. The 3′ end sequence to be added may be derived from, for example, the nopaline synthase or octopine synthase genes, or alternatively from another plant gene, or from any other eukaryotic gene.

As used herein the term “providing a recombinant DNA molecule” may refer to introduction of an exogenous DNA molecule to a plant cell by transformation, optionally followed by regeneration of a plant from the transformed plant cell. The term may also refer to introduction of the recombinant DNA molecule by crossing of a transgenic plant comprising the recombinant DNA molecule with another plant and selecting progeny plants which have inherited the recombinant DNA molecule or transgene. Yet another alternative meaning of providing refers to introduction of the recombinant DNA molecule by techniques such as protoplast fusion, optionally followed by regeneration of a plant from the fused protoplasts.

It will be clear that the methods of transformation used are of minor relevance to the current invention. Transformation of plants is now a routine technique. Advantageously, any of several transformation methods may be used to introduce the nucleic acid/gene of interest into a suitable ancestor cell. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens et al. (1982) Nature 296: 72-74; Negrutiu et al. (1987) Plant. Mol. Biol. 8: 363-373); electroporation of protoplasts (Shillito et al. (1985) Bio/Technol. 3: 1099-1102); microinjection into plant material (Crossway et al. (1986) Mol. Gen. Genet. 202: 179-185); DNA or RNA-coated particle bombardment (Klein et al. (1987) Nature 327: 70) infection with (non-integrative) viruses and the like. In the case of canola or other oilseed rape plants, a suitable transformation method is that disclosed in De Block et al. (Plant Physiol. (1989) 91: 694-701), which disclosure is incorporated by reference herein as if fully set forth.

The recombinant DNA molecules according to the invention may be introduced into plants in a stable manner or in a transient manner using methods well known in the art. The chimeric genes may be introduced into plants, or may be generated inside the plant cell as described e.g. in EP 1339859.

The methods of the invention may be advantageously combined with other oil seed content enhancing methods known from the art. PEP insensitive or less sensitive PPi-PFK can be expressed in combination with other proteins that are known to be effective in oil accumulation be it by increased uptake of substrates, increased provision of precursors of fatty acid synthesis, improved transport of Fatty Acids or acyl-CoAs into the endoplasmatic reticulum, increased enzyme capacity in lipid assembly or reduced lipid catabolism.

The uptake of substrates may be improved by increasing the sugar transport into seed and/or embryo using e.g. AtSUC5 (Baud et al., 2005, Plant J 43: 824-836). Precursors of fatty acid synthesis may be increasingly provided by expression of BASS2NHD1: Na-pyruvate/sodium:proton antiporter—pyruvatc proton symport into plastid (AT2G26900, AT3G19490) (Furumoto et al., 2011, Nature 476: 472-475) or by expression of homomeric acetyl-CoA carboxylase in plastid (AT1G36160, AT1G36180) (Roesler et al., 1997 Plant physiology 113: 75-81) or by increased expression of glycerol-3-phosphate dehydrogenase (Vigeolas et al., 2007, Plant Biotechnol J 5: 431-441); or by increased expression of wrinkled1 (At3g54320) a transcription factor involved in expression regulation of various glycolytic and fatty acid synthesis enzymes (Liu et al. Plant physiology and biochemistry 48: 9-15).

Kim et al. 2013 (. Proceedings of the National Academy of Sciences of the United States of America 110: 773-778) described how the AtABCA9 transporter(AT5G61730) may be used to supply fatty acids for lipid synthesis to the endoplasmic reticulum.

Various publications document increased oil accumulation by increasing the enzyme capacity in lipid assembly. Jain et al., 2000 (Biochem Soc Trans 28: 958-961) described the use of Glycerol-3-phosphate acyltransferase (GPAT) from Safflower and E. coli. Zou et al., 1997 (Plant Cell 9: 909-923) described modification of seed oil content and acyl composition in the brassicaceae by expression of a yeast sn-2 acyltransferase (LPAT) gene. Increased oil accumulation was also achieved by overexpression of diacylglycerolacyltransferase (DGAT) (Jako et al. 2001, Plant Physiol 126: 861-874; Misra et al. 2013 Phytochemistry 96: 37-45) Overexpression of Oleosin has been shown to be effective in green tissues.

Another way of increasing oil accumulation is by reducing lipid catabolism. Kelly et al. (2013) (Plant Biotechnol J 11: 355-361) reported that suppression of the SUGAR-DEPENDENT1 triacylglycerol lipase family during seed development enhances oil yield in oilseed rape (Brassica napus L.) An orthologue of SDP1 in Arabidopsis thaliana is AT5G04040.

There are also reports of expression of such genes in combination. Van Erp et al. (2014) (Plant Physiol 165: 30-36) described that multigene engineering of triacylglycerol metabolism boosts seed oil content in Arabidopsis (overexpression of Wri1 & DGAT1, with suppression of SDP1 (triacylglycerol lipase) in developing seeds). Vanhercke et al. (2013 FEBS Lett 587: 364-369) reported the synergistic effect of WRI1 and DGAT1 coexpression on triacylglycerol biosynthesis in plants.

Accordingly, the invention provides a method for method to increase oil content in seeds or embryos of a Brassica plant comprising the step of preventing feedback inhibition by phosphoenolpyruvate (PEP) of a pyrophosphate-dependent phosphofructokinase (PPi-PFK) present in cells of said seeds or said embryos and the further step of providing said cells with one or more further recombinant DNA molecules comprising the following operably linked DNA fragments:
(d) a plant expressible promoter, preferably a seed-specific promoter;
(e) a DNA region, preferably a heterologous DNA region encoding a polypeptide selected from the following group:
   (i) sucrose transporter capable of influencing sugar transport into the seed or embryo, such as AtSUC5;
   (ii) Na-pyruvate/sodium:proton antiporter;
   (iii) acetyl-CoA carboxylase;
   (iv) glycerol-3-phosphate dehydrogenase;
   (v) transcription factor wrinkled 1;
   (vi) AtABCA9 transporter;
   (vii) Sn-2 acyltransferase
   (viii) lysophophatidic acid acyl transferase
   (ix) glycerol-3-phosphate acyltransferase
   (x) diacylglycerol acyltransferase; or
   (xi) oleosin; and optionally
(f) a transcription termination and/or polyadenylation region functional in plant cells.

In another embodiment, the invention provides a method for method to increase oil content in seeds or embryos of a Brassica plant comprising the step of preventing feedback inhibition by phosphoenolpyruvate (PEP) of a pyrophosphate-dependent phosphofructokinase (PPi-PFK) present in cells of said seeds or said embryos and the further step of providing said cells with a further recombinant DNA molecule comprising the following operably linked DNA fragments:
(c) a plant expressible promoter, preferably a seed-specific promoter;
(d) a DNA region which encodes an inhibitory RNA capable of suppressing expression of sugar-dependent1 triacylglycerol lipase.

Inhibitory RNA as herein used includes antisense RNA, co-suppression (sense) RNA, double stranded RNA including hairpinRNA, siRNA, microRNA and precursors thereof.

Especially suited according to the invention is the combination of seed-specific expression of wrinkled1 in combination with seed-specific expression of a PPi-PFK which is insensitive or less sensitive to feedback inhibition by PEP. A suitable wrinkled polypeptide is that derived from oil palm (SEQ ID No. 5) or a variant protein having at least 90% sequence identity with that amino acid sequence.

According to a second aspect of the invention, prevention of the feedback inhibition of PPi-PFK may be achieved by reducing the steady state level of PEP in cells of Brassica seeds and/or embryos. This can conveniently be achieved by increasing the level or activity of PEP carboxylase and/or PEP carboxykinase in these cells, e.g. by introduction of a transgene encoding PEP carboxylase and/or PEP carboxykinase under control of a seed specific promoter.

Plants according to the invention can be used in a conventional breeding scheme to produce more transformed plants with the same characteristics or to introduce the chimeric gene according to the invention in other varieties of the same or related plant species, or in hybrid plants. Seeds obtained from the transformed plants contain the chimeric genes of the invention as a stable genomic insert and are also encompassed by the invention.

The methods and means described herein are believed to be particularly suitable for Brassica plants. As used herein, a “Brassica plant” is a plant which belongs to one of the species Brassica napus, Brassica rapa (or campestris), or Brassica juncea. Alternatively, the plant can belong to a species originating from intercrossing of these Brassica species, such as B. napocampestris, or of an artificial crossing of one of these Brassica species with another species of the Brassicaceae (Cruciferacea). As used herein “oilseed plant” refers to any one of the species Brassica napus, Brassica rapa (or campestris), Brassica carinata, Brassica nigra or Brassica juncea.

It is expected however that the methods of the invention can also be applied to other oil producing plants such as flax (Linum usitatissimum), safflower (Carthamus tinctorius), sunflower (Helianthus annuus), maize or corn (Zea mays), soybean (Glycine max), mustard (Brassica spp. and Sinapis alba), crambe (Crambe abyssinica), eruca (Eruca saiva), oil palm (Elaeis guineeis), cottonseed (Gossypium spp.), groundnut (Arachis hypogaea), coconut (Cocus nucifera), castor bean (Ricinus communis), coriander (Coriandrum sativum), squash (Cucurbita maxima), Brazil nut (Bertholletia excelsa) or jojoba (Simmondsia chinensis) gold-of-pleasure (Camelina sativa), purging nut (Jatropha curcas), Echium spp., calendula (Calendula officinalis), olive (Olea europaea), Lesquerella spp., Cuphea spp., meadow foam (Limnanthes alba), avocado (Persea Americana), hazelnut (Corylus), sesame (Sesamum indicum), safflower (Carthamus tinctorius), tung tree (Aleurites fordii), poppy (Papaver somniferum).

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

As used herein “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A chimeric gene comprising a DNA region which is functionally or structurally defined, may comprise additional DNA regions etc.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification arc herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R.D.D. Croy, jointly published by BIOS Scientific Publications Ltd (UK) and Blackwell Scientific Publications, UK. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR-Basics: From Background to Bench, First Edition, Springer Verlag, Germany.

Throughout the description and Examples, reference is made to the following sequences:
SEQ ID No. 1: amino acid sequence of PPi-PFK from Thermoproteus tenax.
SEQ ID No. 2: amino acid sequence of PPi-PFK from Naegleria fowleri.
SEQ ID No. 3: amino acid sequence of PPi-PFK from Methylococcus capsulatus.
SEQ ID No. 4: amino acid sequence of PPi-PFK from Amycolatopsis methanolica.
SEQ ID No. 5: nucleotide sequence of the codon-optimized coding region for WRI1 of Elaeis guineeis.
SEQ ID No. 6: amino acid sequence of WRI1 of Elaeis guineeis.
SEQ ID No. 7: nucleotide sequence of the oleosin promoter.

### EXAMPLE 1

### Quantitative Analysis of Seed Metabolism Indicates Allosteric Control Mechanisms in Brassica napus

Seeds develop by absorbing nutrients from their mother plant, and using these to synthesize a combination of starch, protein and lipid. The size and number of seeds which finally develop determines the crop's yield, while their composition determines the end-use quality of the crop. The conversion of nutrients into storage products involves a complex network of metabolic reactions, many of which are subject to transcriptional, translational and post-translational regulation. Attempting to engineer seed composition clearly requires a firm understanding of these regulatory networks.

The seed's central metabolism differs markedly from that of either a photosynthesizing leaf or root. In most species, the immature seed is green for a period during its development, so during this phase is regarded as being photoheterotrophic. A further level of complexity arises as a result of spatial heterogeneity within the seed which generates a significant degree of metabolic heterogeneity. Thousands of genes are involved in seed development. In Arabidopsis thaliana, about 17,500 distinct mRNAs (˜60% of the full gene complement) are transcribed in the seed during its development, with about 1,300 of these being seed-specific. While this set of genes no doubt provides a full picture of the stage- and tissue-specific framework of gene activity, many examples have been described where the pairs of transcript versus protein or protein versus flux are rather discordant. Thus, monitoring of gene expression is not sufficient to define metabolic activities in vivo.

Post-transcriptional regulation seems to be of particular relevance for the control of metabolic flux. Mechanisms in play are protein modification, allosteric enzyme regulation and the control of substrate availability. A systems level approach is needed to unravel such a level of complexity. A suggested start involves metabolic modelling to provide a quantitative view. In microbes, this approach has already helped identify targets for molecular engineering and to suggest improved engineering strategies. The current state-of-the art is attempting to combine in silico prediction with synthetic biology. The extension of this form of analysis to plants is expected to drive improvements in the formulation of effective metabolic engineering strategies and new ways of breeding crops.

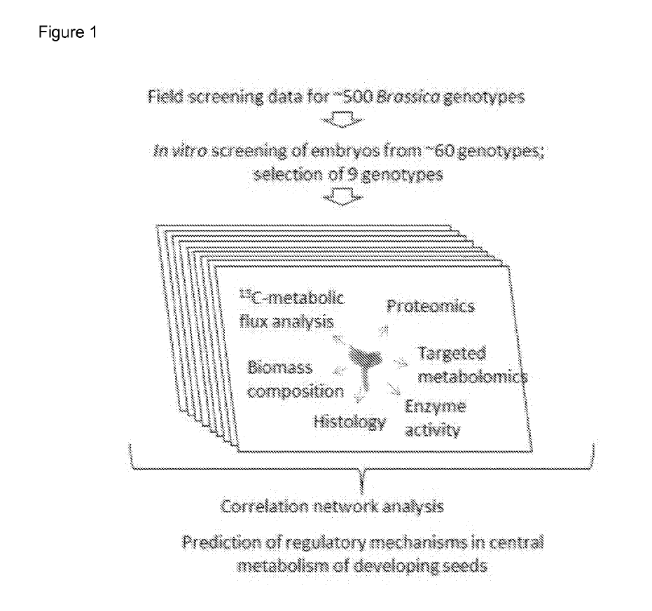

Here, a combination of targeted metabolomics, proteomics, enzyme activity profiling and 13C-based metabolic flux analysis (MFA) is described to understand the central metabolism of the developing seed of oilseed rape (Brassica napus), a leading temperate oilseed crop. In particular, the aim was to explore how this metabolism was regulated over and above the usual level of transcriptional control. Given the practical difficulties associated with extending a high intensity systems biology approach to several hundred accessions, an initial screen was conducted to identify a diverse panel of materials which contrasted with respect to their embryo composition. The experimental strategy taken (FIG. 1) was based on the outcomes of a previous field trial where the performance of a selection of ˜500 accessions was documented; these data led to the selection of a panel of ˜60 entries varying widely with respect to their seed composition and weight. A second screen was applied to identify a subset of entries showing contrasting embryo growth rates and biomass compositions. Finally, the choice focused on seven entries, to which were added a transgenic line engineered to over-express a gene encoding the enzyme diglyceride acyltransferase 1 (DGAT1) and its progenitor cultivar (b144). The feature of the transgenic line was that as DGAT1 catalyses the formation of triglycerides from diacylglycerol and acyl-CoA, the transgenic plant's seed develop an elevated lipid content. The dataset acquired from various analyses of these nine lines has been used to identify regulators and markers of seed metabolism.

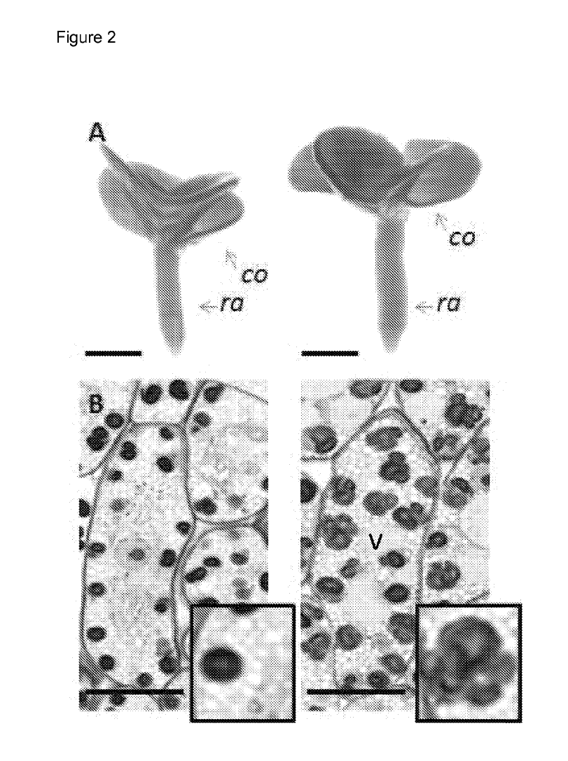

In vitro screening identified entries contrasting for growth rate and embryo composition. The dissected embryos, cultured under uniform conditions, of the 63 oilseed rape entries were similar with respect to their morphology, but varied in size. Histological analysis revealed that the cytoplasm of the plastids present in the small embryos typically contained one or two small starch grains, along with numerous lipid bodies (FIG. 2). In contrast, the plastids in the cytoplasm of large embryos harbored large aggregates of starch grains, along with a substantial vacuole and few and smaller lipid bodies. A subsequent analysis of fresh and dry weights, total lipid content, fatty acid composition and total protein content confirmed the presence of variation in embryo composition. Based on contrasting embryo weight, lipid content and lipid/protein ratio, seven entries were selected to represent the range of variation present in oilseed rape. Their embryos, along with those of the DGAT1 over-expressing transgenic line and its progenitor cultivar b144, were cultured in bulk in three replicated sets to provide sufficient material for subsequent analyses.

Variation in the efflux into biomass components was correlated with fluxes through alternative pathways. The embryos of all nine test entries were cultivated in the presence of 13C-labeled glucose. The greatest variability in embryo composition related to starch content, which ranged from 3% and 22% on a dry weight basis; the range in lipid content was 25-37%, and in protein content 14-23%. The starch content was negatively correlated with both that of lipids (R=−0.90) and protein (R=−0.73), suggesting that variation in embryo composition, at least across the nine test entries, reflected a trade-off between starch and lipids/protein. To assess flux control over embryo composition, those reactions for which the flux was correlated with one or more of lipids, protein and starch were identified. The fluxes were determined using a 13C-based MFA, where after mass spectrometric analysis of mass isotopomer signatures in various isolated metabolites, the flux distribution in central metabolism is estimated based on isotopomer network simulation and flux parameter fitting.

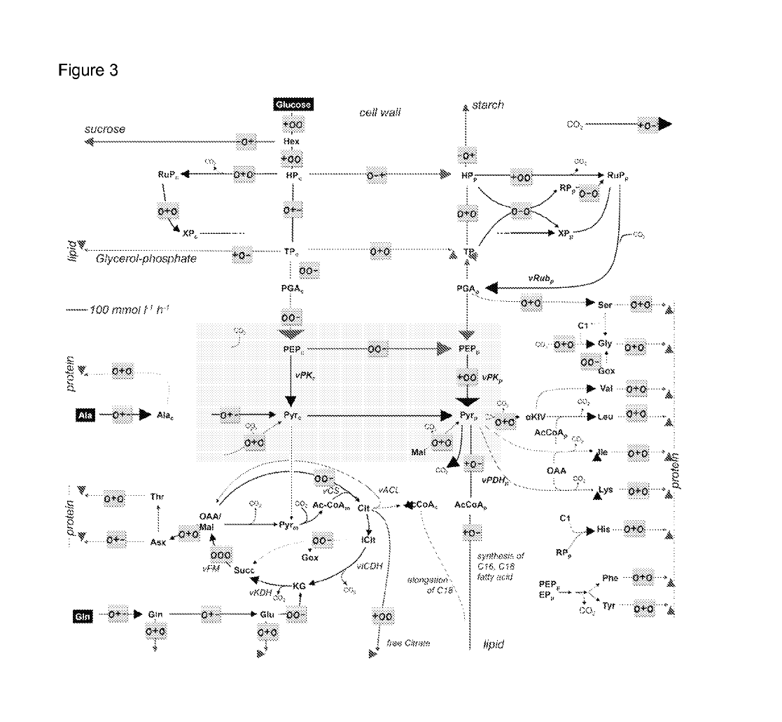

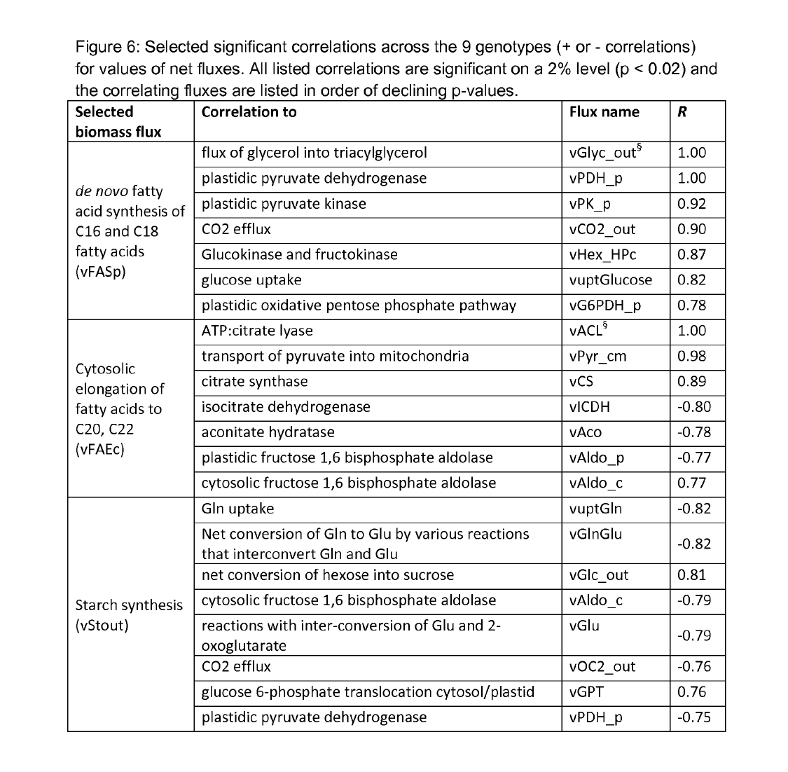

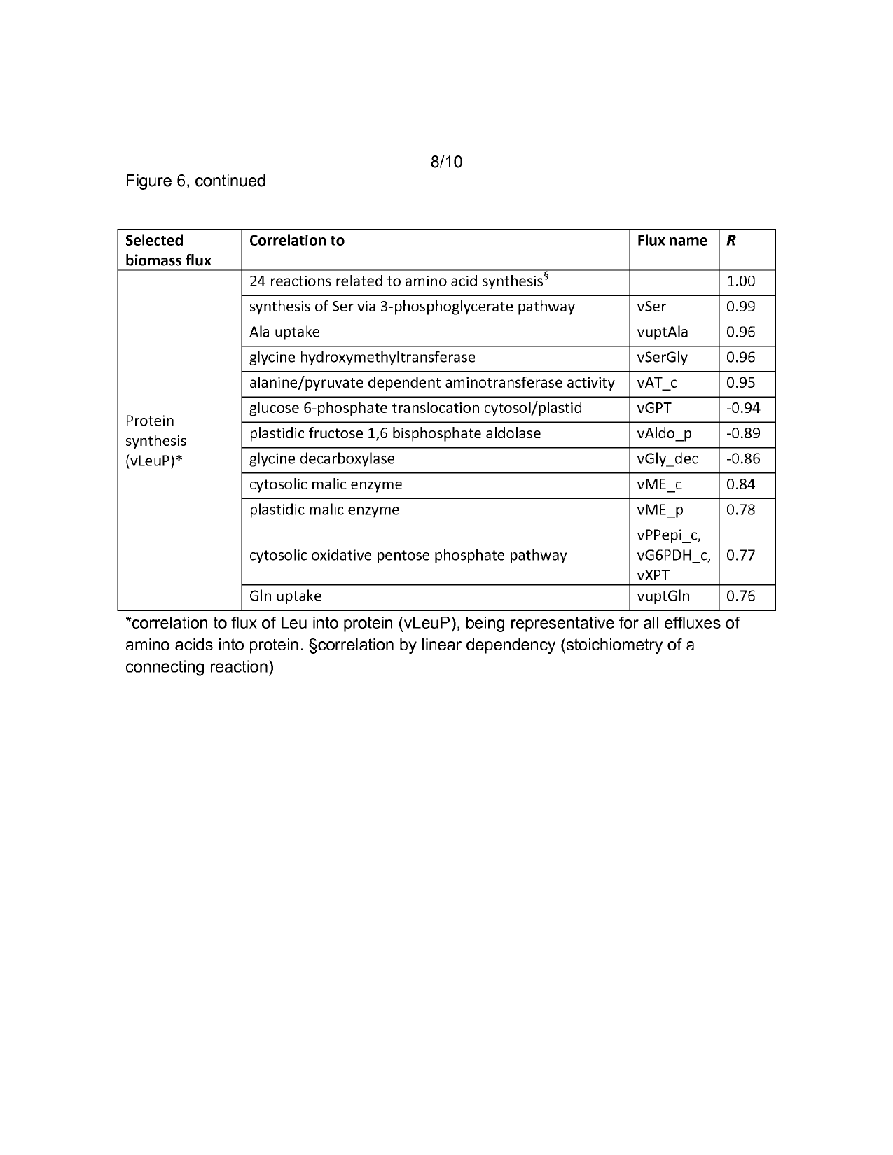

A representative flux distribution in the central metabolism of cv. b144 is shown in FIG. 3, along with information regarding correlations (p≦5%) with the synthesis of lipid, protein or starch. The most significant such correlations (p≦2%) are listed in FIG. 6. Fatty acid synthesis was mostly fed by the catabolic conversion of sugars to pyruvate (FIG. 3). The uptake of glucose (vuptGlucose) was positively correlated with the flux into fatty acids. Between hexose phosphates (HP) and phosphoenolpyruvate (PEP) the flux map shows carbon flux to be distributed via cytosolic glycolysis and the ribulose 1,5 bisphosphate carboxylase (RubisCO) shunt (vRubp). A preference for cytosolic over plastidial glycolysis was common to all entries and has been proposed from an analysis of transcriptomic data. Apart from glycolysis and the RubisCO shunt, hexose phosphates (HPs) can be consumed by the oxidative pentose phosphate pathway (OPPP), which generates NADPH for reductive biosyntheses. Across the nine genotypes the cytosolic fluxes within the OPPP (vG6PDHc) were about one order of magnitude higher than obtained in the plastidial one. vG6PDHp was positively correlated with plastidial fatty acid synthesis, indicating its importance for lipid synthesis. However, only between 0% and 10% of the NADPH demand for fatty acid synthesis was accounted for by the oxidative decarboxylation of glucose 6-phosphate in the plastid.

In the lower part of glycolysis, PEP can be converted to pyruvate (Pyr) via either plastidic or cytosolic pyruvate kinase (PKp or PKc) (FIG. 3) or, less directly, by the carboxylation of PEP to form oxaloacetate, with a subsequent reduction and oxidative decarboxylation effected by, respectively, malate dehydrogenase and malic enzyme (see highlighted sub-network in FIG. 3). Across the nine entries, only 4-7% of the PEP was converted into lipids via the latter route, while 50-70% of the plastidial pyruvate was converted to pyruvate via PKp. Both PKp flux and enzyme activity (see below) were positively correlated with the flux into fatty acid synthesis (FIG. 3, FIG. 6), suggesting that PKp exerted a strong measure of control over fatty acid synthesis and lipid content.

Apart from the de-novo synthesis of fatty acids in the plastid, C18 fatty acids can also be elongated to C20 or C22 chains via a cytosolic elongation system which requires acetyl-CoA derived from citrate by a cytosolic ATP:citrate lyase activity (FIG. 3). There was substantial variability for the fatty acid composition and flux into the fatty acid cytosolic elongation (vFAEc) across the nine entries. The derived correlations (FIG. 6) indicated that the increased vFAEc was achieved by a concomitant increase in citrate synthesis, that is, the higher transfer of pyruvate from cytosol to the mitochondria (vPyr_cm) was achieved by the up-regulation of mitochondrial pyruvate dehydrogenase and citrate synthase (vCS), as well as by the decreased consumption of citrate due to aconitase/isocitrate dehydrogenase (vICDH) activity. The TCA cycle-related fluxes were all relatively small: while fatty acid synthesis flux ranged between 96 and 133 mmol/L/h, the absolute values for vCS, vFM, vICDH and vKDH ranged between 0.1 and 13.6 mmol/L/h. Fluxes through the mitochondrial, cytosolic and plastidial isoforms of malic enzyme (vMEm, vMEc, vMEp) were similarly small.

The peak catalytic activity of most enzymes was not correlated with the embryo components and was well above the necessary pathway flux. The extent to which the metabolic status (as measured by 13C-MFA) and the embryo composition were correlated with enzyme activity was determined by estimating the total extractable activity (Vmax) of 26 key metabolic enzymes. The activity of four glycolyis-associated enzymes (PEP carboxylase, PEP carboxykinase, PKp and pyrophosphate-dependent phosphofructokinase) and that of aspartate transaminase was positively correlated with the proportion of lipid present in the embryo; that of fructokinase was positively correlated with the protein content; but none were associated with either starch or cell wall material accumulation.

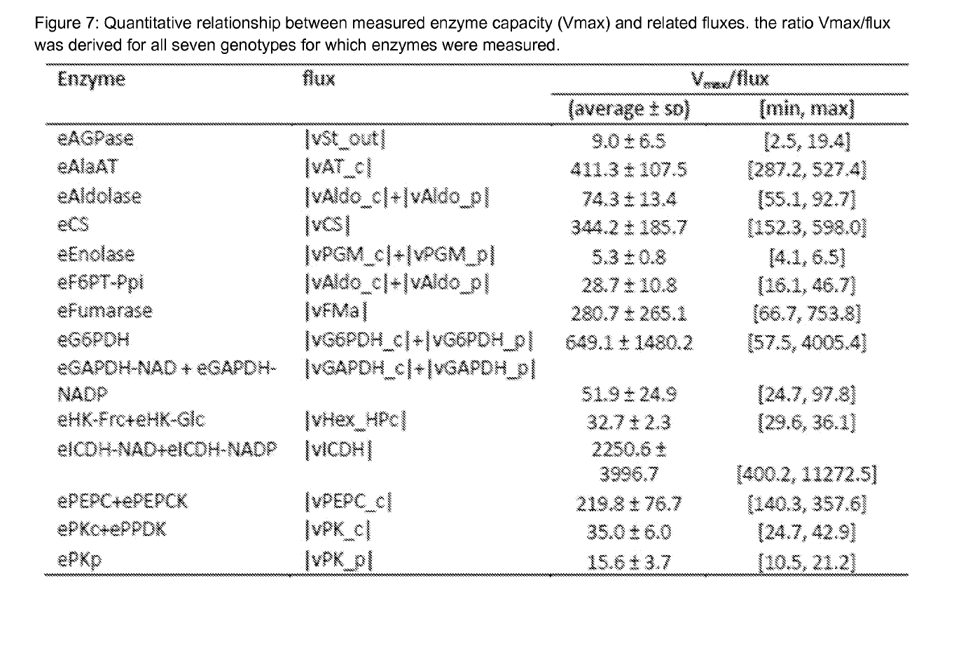

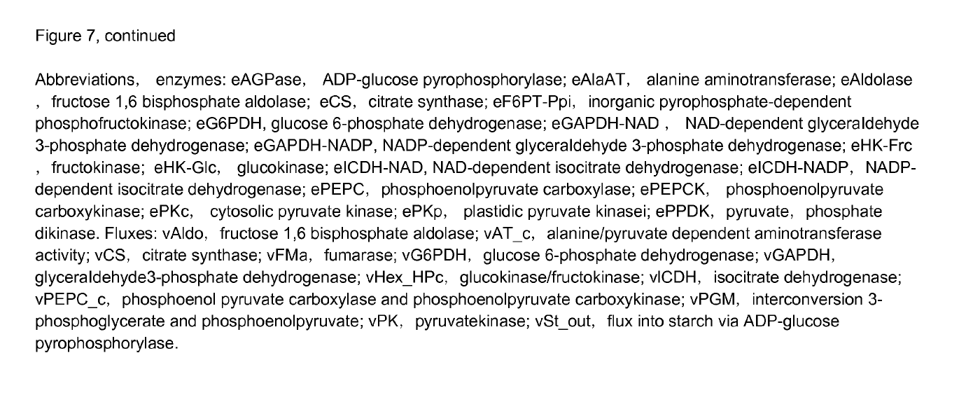

Enzyme activities and fluxes were compared after first scaling to comparable units (FIG. 7). Since the enzymes were assayed under conditions of substrate saturation, the values represented an estimate of their peak catalytic capacity (Vmax). In several cases where (e.g. for enolase) the assay captured both plastidial and cytosolic activity, the absolute values of fluxes were summed. For enolase and ADP-glucose pyrophosphorylase (AGPase), the Vmax/flux ratio was close to unity, but for the other enzymes, the activity level was substantially above the steady state flux. For the set of citrate cycle enzymes (fumarase, citrate synthase, isocitrate dehydrogenase), Vmax was two to three orders of magnitude higher than the steady state flux, while for the PEP carboxylase and PEP carboxykinase, it was around 70 fold higher.

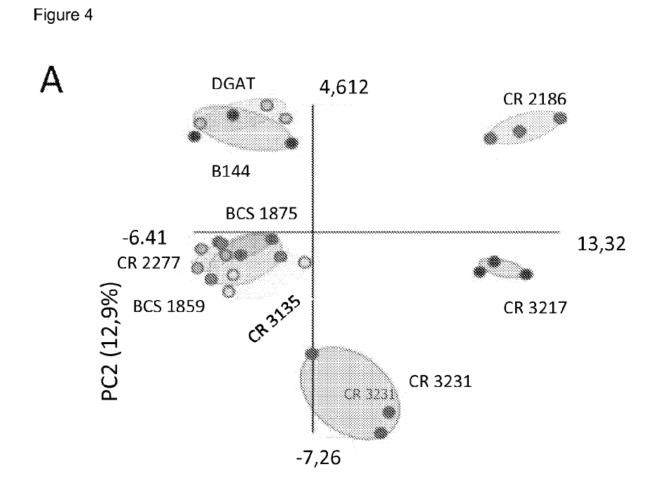

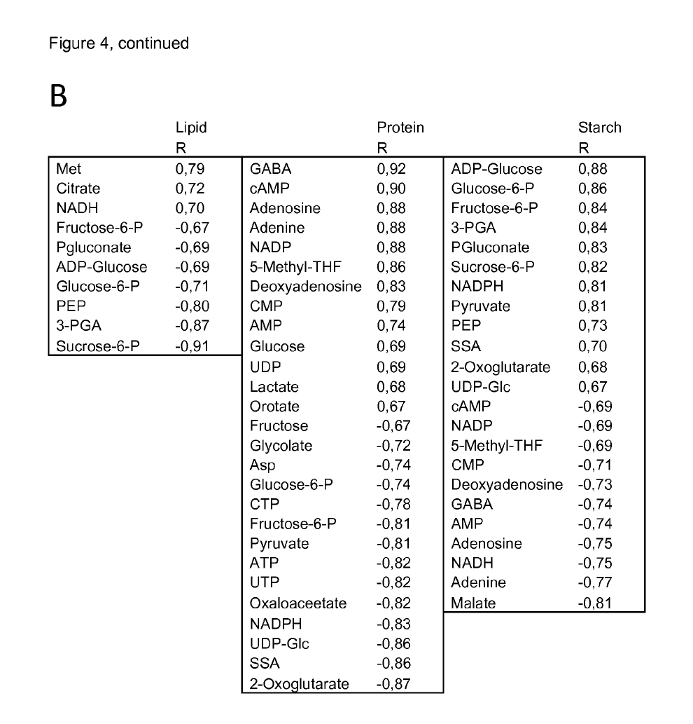

Identification of metabolites diagnostic for embryo components. The metabolic profiles of the nine entries were distinct. The use of the LC/MS platform enabled the quantification of about 90 metabolite intermediates. A principal component analysis identified only minor differences between the DGAT transgenic line and its progenitor cultivar, while differences were very apparent within the set of (genetically more diverse) seven entries (FIG. 4A). For example, entry 2277 accumulated rather high levels of lipid/protein but overall less dry matter than entry 3231, which in turn featured a marked reduction in the content of hexose-6-phosphates, several glycolytic intermediates, most free amino acids and various other metabolites. A correlation analysis was used to identify which metabolites were most strongly associated with the accumulation of lipid, protein and starch. For lipid content, the most significant correlations involved sucrose-6-phosphate, a number of glycolytic intermediates (PEP, 3-phosphoglycerate (3-PGA), pyruvate) and hexose phosphates (FIG. 4B); for protein content, the analysis identified γ-aminobutyric acid, the signaling molecule cyclic 3′,5′-adenosine monophosphate (cAMP) and several nucleotides and cofactors; finally, for the starch content, the candidates were its direct precursor ADP-glucose and hexose-6-phosphates.

The intermediates PEP and 3-PGA are known to allosterically affect enzymatic activity of ATP-dependent PFK and AGPase, respectively. We checked if this effect, described to be active in the leaf, also takes place in the oilseed rape embryo, and found clear activity changes.

By relating the concentration of metabolites to the relevant flux values, a calculation was made of the various turnover times. This analysis indicated that the hexose phosphates (glucose-1 phosphate, glucose-6 phosphate, fructose-6 phosphate, fructose-1,6 diphosphate) were consumed within 10-50 min; turnover times were positively correlated with starch content, but negatively to lipid content. The same relationships applied to glycolytic intermediates, but with much shorter consumption times (PEP: 44 s, 3-PGA: 4 min, pyruvate: 7 min). These tendencies might indicate that lipid biosynthesis responds faster and/or is more sensitive to precursor limitations than starch biosynthesis.

The quantification of metabolite levels further allowed the mass-action ratio (the ratio between the in vivo concentration of the product and that of its substrate) to be calculated for individual enzymatic reactions. These were then compared with their respective equilibrium constant Keq (the ratio between the product and the substrate concentrations when the reaction was at thermodynamic equilibrium and there was no net flux) to reveal how far each reaction was displaced from its equilibrium. (A reaction can be regarded as irreversible when the mass-action ratio has been displaced from its Keq by a factor of >10, Tiessen et al, 2012). The outcomes indicated that both sucrose cleavage mediated by sucrose synthase, and hexose mobilization mediated by gluco/fructokinases were essentially irreversible in vivo in the oilseed rape embryo. The same applied with respect to both the following and the final glycolytic steps, catalyzed by, respectively, phosphofructokinase (PFK) and pyruvate kinase, as well as starch synthesis (mediated by AGPase). In contrast, various sugar conversion steps (mediated by phosphoglucomutase, phosphoglucose isomerase and UDP-glucose pyrophosphorylase) were readily reversible. Note that these calculations did not account for the non-homogeneous sub-cellular distribution of metabolites.

A proteomic comparison between low- and high-lipid entries highlighted several post-translational modifications but, overall, indicated that the level of synthetic enzymes present was unlikely to underlie differences in synthetic flux and as a result, little influence on the lipid content of the embryo.

The flux distribution based on 13C-MFA is represented in FIG. 3. The conversion of sugars into lipids involved glycolysis, the operation of the RubisCO shunt and the minimization of flux through the TCA cycle. At the same time, flux through the NADPH-generating OPPP appeared to be small relative to the NADPH demand of various synthetic reactions. Plastidial fatty acid synthesis is fed in large part through plastidial pyruvate kinase (PKp), while the parallel route via the cytosol provides smaller quantities of pyruvate. Across the germplasm analysed here, increased lipid accumulation was correlated with an increased PKp flux and enzyme activity. While glycolytic activity increased with lipid content, the accumulation of pathway intermediates (glucose-6-phosphate, fructose-6-phosphate, 3-PGA, PEP) was negatively correlated with the lipid content (FIG. 4B), which suggested that an allosteric feedback regulation of glycolysis may contribute to the control of storage lipid synthesis. Bottom-up regulation has been established as a major control mechanism for plant glycolysis; the notion is that the control of glycolytic flux is exerted via a feedback inhibition of ATP-dependent PFK. PFK is strongly inhibited by PEP, while the potent activation of pyrophosphate-dependent phosphofructokinase (PFP) by fructose-2,6-bisphosphate is strongly diminished by PEP. An increased activity of the PEP-consuming enzymes PK or PEP carboxylase can reduce the PEP concentration, which in turn relieves the feed-back inhibition of PFK, allowing for an increased pathway flux. Both PFK and PFP were expressed in developing oilseed rape embryos. The activity of PEP carboxylase, PEP carboxykinase and PKp was positively correlated with lipid accumulation, while the level of PEP and other glycolytic intermediates was negatively correlated. These relationships suggested that allosteric control mechanisms are relevant in the control of glycolytic flux and therefore in plastidial fatty acid synthesis in oilseed rape. Note that in A. thaliana, a model plant very closely related to oilseed rape, the expression of PKp in the seeds has been identified as being under the control of WRI1, a transcription factor known to be a “master regulator” of the conversion of sucrose into fatty acids. The present study has extended this concept to the control of lipid synthesis, in that the upper reactions of glycolysis are under allosteric feedback control mediated by PEP. In addition, PFP activity has been seen to correlate positively with lipid accumulation, which can be expected to work synergistically to the PEP-mediated flux control.

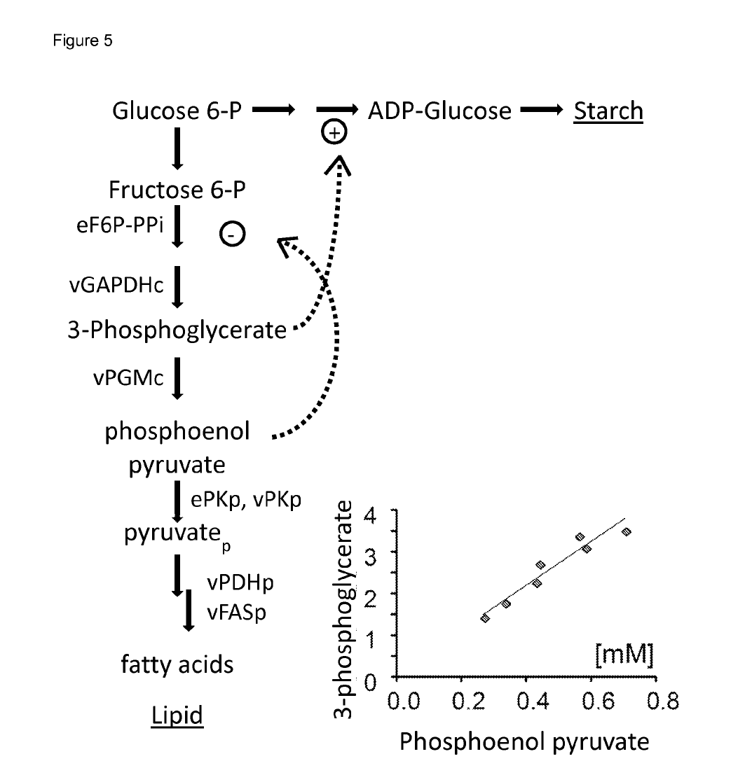

In the context of the observed lipid/starch trade-off, the flux control of glycolysis by PEP can be extended to the control of starch synthesis. Starch is synthesized in plastids from ADP-glucose, which is positively correlated to starch content (FIG. 4B). ADP-glucose is formed by AGPase, an enzyme representing an important control step in the synthetic pathway and allosterically activated by 3-PGA. According to our model (FIG. 5) the concentrations of the two glycolytic intermediates PEP and 3-PGA (highly correlated with one another; see insert in FIG. 5), likely control glycolysis and starch synthesis: rising concentrations promote starch synthesis but repress glycolytic flux, while falling concentrations (e.g. due to high rates of fatty acid synthesis) shift carbon partitioning toward glycolysis. Changes in the activity of PKp and PEP carboxylase can modulate the level of both PEP and 3-PGA.

The proposed coordinated bottom-up control of lipid and starch synthesis is supported by studies on PKp mutations in A. thaliana, in which mature seeds carrying defective PKp subunits produce much more starch than do wild type ones, and also accumulate more PEP and pyruvate. The latter is consistent with the idea of swelling of PEP levels in response to reduction in PKp. The present findings bear out what is a well-known regulatory mechanism, but in unprecedented detail. Based on an integrated and quantified analysis of metabolite levels, enzymes and fluxes, the relevance of this regulatory machinery in carbon partitioning and lipid synthesis in the seed has been much more clearly elucidated.

Along with the suggested allosteric control mechanism with PEP/PGA at its heart, it is recognized that glycolysis takes place both in the cytosol and the plastids. The flux model implies that a large proportion of the carbon flux enters the plastids in the form of PEP. The PEP/phosphate translocator (PPT) in vivo appears reversible and modulation of plastidial PEP levels by PKp can be propagated across the chloroplast envelope. PPT has been ascribed an important role in lipid synthesis, and its over-expression in tobacco seeds has been demonstrated to promote lipid accumulation.

At the proteome level, the major difference between the high and low lipid entries was accounted for mainly by storage proteins and protein metabolism. Surprisingly, there was no evidence for any coordinated up/down-regulation of the central metabolic pathways, and entry-to-entry variation only concerned two metabolic enzymes. The major discordance between metabolic flux, Vmax and enzyme abundance suggests that fluxes in the seed central metabolism were not significantly regulated at the level of transcription/translation, but rather at the post-translational level (e.g. allosteric control exerted by the PEP/3-PGA motif). Transcriptional control is especially improbable where Vmax values arc orders of magnitude greater than the steady state flux, which in the oilseed rape embryo, was clearly the case for the TCA cycle enzymes (FIG. 7). Metabolic flux control exerted via changes in enzyme concentration is costly and relatively slow. In contrast, post-translational flux control may allow the seed to rapidly adjust fluxes to reflect changes in substrate availability (for example, being much higher during the daylight hours than during the night). Transcriptional control in the seeds is associated with stage-specific maximum metabolic capacity, while flux control in the central metabolism seems rather regulated by substrate availability, allosteric control and/or post-translational modification. The latter is supported by the observation that most glycolytic and TCA cycle enzymes present in the seed are phosphorylated and/or acetylated. Some enzyme protein modifications were also noted in the present materials. Similar conclusions on flux control in central metabolism were recently drawn for microorganisms.

The accumulation of a number of metabolites has been shown to be highly correlated with that of the various embryo constituents (FIG. 4B), and no known regulatory function has been associated with many of them to date. These include sucrose-6-phosphate, an intermediate of sucrose re-synthesis. Its fairly low concentration (pmol/mg range) in the oilseed rape embryo corresponds to that typically measured for signaling compounds. Defining the role of such intermediates requires a large-scale and systematic analysis of metabolite-protein interactions. We further noticed that the presence of cAMP was positively correlated with protein storage; it is well-established as a secondary messenger in both microorganisms and animals, and has been shown to be involved in metabolic flux control. Several plant ion channels and thioesterases are known to possess cyclic nucleotide binding domains, associated with a wide range of physiological responses. The function of cAMP in the plant cell is still unclear, but the strong correlation of its accumulation with protein storage activity suggests a hitherto unknown mechanism of metabolic flux control.

Since control over flux through a metabolic pathway can be considered from the standpoint of its overall thermodynamic organization, substrate-product ratios and enzyme equilibrium conditions are also of interest. The present calculations indicated that both the entry (fructo-/glucokinase, PFK) and final exit (PK) steps of glycolysis were essentially irreversible, consistent with the situation pertaining in other sink tissues such as the cereal caryopsis and various starch-storing tubers. Since the majority of glycolytic reactions are assumed to operate close to their equilibrium, net flux/direction can be effected by even a small change to either the substrate or the product concentration. In the oilseed rape embryo, the PEP-producing enzyme enolase had a Vmax/flux ratio close to unity, and its product (PEP) had a fast consumption rate (44 s). Lipid synthetic fluxes were clearly correlated with the level of both PEP and PGA, while the PEP/PGA-ratio was consistent across the entries. Consequently, even a modest increase in PEP-consuming flux (e.g. via PEPC or fatty acid synthesis) would be expected to instantly promote enolase activity. The flux, propagated via mass action through the metabolic network, would serve to stimulate carbon flow along the glycolytic pathway. High lipid entries, which have a more elevated demand for glycolytic intermediates (such as the fatty acid synthesis precursors PEP and pyruvate), could stimulate this flux by effectively removing the intermediates. The capacity for high lipid storage can be expected to depend on both the embryo's ability to catalyze high glycolytic flux (the Vmax of several glycolytic enzymes was noticeably heightened in high lipid entries), and its high metabolic demand, as manifested by the rapid withdrawal of fatty acids and their precursors via the relevant biochemical and transport reactions.

The application of an integrated biology approach has derived a model for flux regulation in the seed central metabolism of an important oil crop. The analysis has shown that the Vmax of glycolytic, TCA cycle and other pathway enzymes seldom corresponded with either embryo composition or intracellular flux, while the levels of several metabolites (substrates, products and effectors) involved in the regulation of enzyme activity was quite variable. The hypotheses which arise are that (1) increases in flux do not necessarily need shifts in enzyme protein abundance effected by transcriptional/translational control, (2) the flux capacity (enzyme concentration) is mostly in excess, and (3) metabolic regulation occurs to a significant extent via allosteric control. The PEP/3-PGA-model (FIG. 5) suggests how carbon flow underlying seed metabolism can be adjusted to the constraints imposed both by assimilate supply and demand. As flux regulation is propagated via mass action through the metabolic network, changes in both supply and demand can both be drivers of flux changes.

### Materials and Methods

### Plant Growth and Procedure for In Vitro Screening

Plants of oilseed rape (Brassica napus) were grown in phytochamber at 18° C. with 16 h of light (400 μmol quanta m-2 s-1) and a relative air humidity of 60%. At the time of flowering, plants were tagged for determination of developmental stages. At 20 days after flowering, intact embryos were isolated, and kept in liquid culture for 10 days under photoheterotrophy (50 μmol quanta m-2 s-1) at 20° C. with organic nitrogen sources according to previously established protocols (Schwender et al, 2006. J Biol Chem 281: 34040-34047). Embryos from each genotype were grown in 3 independent batches. After 10 days of culture, embryos were weighed, freeze-dried and weighed again for determination of fresh and dry weight, respectively. Embryo material was pulverized and used for the quantification of total lipid content (using TD NMR as in Fuchs et al, 2013 Plant Physiol 161: 583-593), fatty acid composition (using gas chromatography as in Borisjuk et al, 2013 Plant Cell 25: 1625-1640) and total protein content (measured as total nitrogen*5.64 using elemental analysis as in Borisjuk et al, 2013). This in-vitro system has been used in former studies to describe flux distribution and pathway usage in central metabolism, which demonstrated that major aspects of in planta seed metabolism can be mimicked in vitro (Schwender et al, 2003 J Biol Chem 278: 29442-29453, 2004 Nature 432: 779-782, 2006 supra; Schwender, 2008 Curr Opin Biotech 19: 131-137).

### In-vitro Culture of Embryos and 13C Metabolic Flux Analysis of Selected Genotypes

Brassica napus embryos of 9 genotypes (b144, DGAT, BCS1875, CR2277, CR3231, CR2186, CR3217, CR3135, BCS1859) were dissected aseptically about 20 days after flowering and grown in a liquid medium at 20° C. under continuous light (50 μmol m-2 sec-1). Cultures were kept in tissue culture flasks with vented seal cap (CytoOne T75 #CC7682-4875, USAScientific, Ocala, Fla., USA), containing 13 ml of liquid medium and four embryos per flask. The liquid growth medium contained 20% (w/v) polyethylene glycol 4000 and the carbon and nitrogen sources Glucose (120 mM), Gln (35 mM), and Ala (10 mM). Labeling experiments contained unlabeled Glucose (96 mM) as well as [U-13C6]Glucose (12 mM) and [1,2-13C2]Glucose (12 mM). Inorganic nutrients were used similarly to Schwender et al (2003, supra): CaCl2 (5.99 mM), MgSO4 (1.5 mM), KCl (4.69 mM), KH2PO4 (1.25 mM), Na2EDTA (14.9 mg L-1), FeSO4.7H2O (11.1 mg L-1), H3BO3 (12.4 mg L-1), MnSO4H2O (33.6 mg L-1), ZnSO4.7H2O (21 mg L-1), KI (1.66 mg L-1), Na2MoO4.2H2O (0.5 mg L-1), CuSO4.5H2O (0.05 mg L-1), COCl2.6H2O (0.05 mg L-1), nicotinic acid (5 mg L-1), pyridoxine HCl (0.5 mg L-1), thiamine HCl (0.5 mg L-1), folic acid (0.5 mg L-1). pH was adjusted to 5.8 using KOH. Growth medium was sterilized by 0.22-mm sterile vacuum filter units (Stericup; Millipore). After 10 days of culture, embryos were harvested, rapidly rinsed with NaCl solution (0.33 M), and after determination of fresh weight embryos were frozen in liquid nitrogen and stored in −80° C. Experiments using unlabeled substrates were done in 3 replicates to determine growth kinetics and biomass composition. Experiments using labeled substrates were done in 4 replicates to determine metabolic fluxes. Flux analysis was performed using the 13CFLUX2 toolbox (Weitzel et al., 2013 Bioinformatics 29: 143-145). The network of central metabolism is defined by 14 free net fluxes as well as 21 biomass effluxes which are derived from biomass composition of the different genotypes, and growth rate. Uptake fluxes of glucose, Ala and Gln, as well as the net efflux of CO2 into the environment, however, depend on isotope tracer based flux parameter fitting like the steady state fluxes in central metabolism. An initial validation of the flux parameter fitting results can therefore be made by assessing the Carbon Conversion Efficiency (CCE), given by:

((total carbon uptake flux)−(carbon efflux))/(total carbon uptake flux),

based on uptake fluxes of glucose, Gln and Ala and CO2 net efflux (FIG. 3). The resulting CCE values ranged genotype-specific between 79% and 86%, which is in good agreement with the value of 86% determined before by experimental carbon mass balance of medium substrates, CO2 emission and biomass formation for B. napus embryos cultured under similar photoheterotrophic conditions (Goffman et al., 2005 Plant Physiol 138: 2269-2279).

Statistical evaluation of flux values was done by repeated random re-sampling of the MS data and flux measurements according to the measurement standard deviations and re-determination of fluxes by random-start optimization. Based on the so obtained standard deviations (SD) of fluxes we assess the statistical quality of the all fluxes as follows: For a given flux the SD be smaller than 10% of the largest absolute value across all net fluxes and genotypes. For a given flux this criterion has to hold across all genotypes. Accordingly, 7 fluxes (vGAPDH_c, vGAPDH_p, vGPT, vPGM_c, vPGM_p, vPPT, vTPT) were found to be not well determined. These constitute parts of the two parallel sections of glycolysis in the cytosol and the plastid compartment.

### Metabolite Extraction and Analysis

Frozen embryo material was extracted and analyzed using liquid chromatography (LC) coupled to mass spectrometry (MS). Compound identities were verified by mass and retention time matches to authenticated standards. External calibration was applied using authenticated standards.

### Enzyme Assays

Enzymes were extracted from 20 mg aliquots of frozen ground leaf tissue by vortexing and mixing in 500 μl of common extraction buffer [10% (v/v) glycerol, 0.25% (w/v) BSA, 0.1% (v/v) Triton X-100, 50 mM Hepes/KOH, pH 7.5, 10 mM MgCl2, 1 mM EDTA, 1 mM EGTA, 1 mM benzamidine, 1 mM ε-aminocapronic acid, 1 mM phenylmethylsulfonyl fluoride (PMSF), 10 mM leupeptin, 1 mM dithiothreitol (DTT)] and c. 10 mg polyvinyl pyrrolidone. PMSF was added just before extraction. DTT was omitted when using peroxidase-based or MTT-based indicator reactions (MTT: methylthiazolyldiphenyl-tetrazolium bromide). A 96-head liquid handling robot (Evolution P3, Perkin Elmer, Wellesey, Mass., USA) was used to perform subsequent dilutions of these initial extracts and to load extracts on to 96-well micro plates. During this process micro plates were maintained at 4° C. using peltier cooled blocks. These micro plates were then transferred to a second robot platform (Microlab STAR, Hamilton, Reno, Nev., USA) which enabled simultaneous initiation and termination of reactions in all 96-wells, an automated incubation station (Automated Hotel, Inheco, Munich, Germany) enabled controlled incubation of reactions. Reactions were started with the addition of a substrate or co-factor and incubated at 30° C. for 20 min. Reactions were then stopped using 0.5M HCl, 0.5M NaOH or 80% ethanol. The concentration of the products of these stopped reactions (NAD+, NADH, NADPH or glycerol-3-phosphate) was then determined using cycling assays. The individual stopped assays and the subsequent cycling assays are described in depth elsewhere (Gibon et al, 2004 Plant Cell 16: 3304-3325; Gibon et al, 2002 Plant J 30: 221-235). Nitrate reductase and glutamine synthetase were determined using end point assays (Gibon et al, 2004, supra). Cytosolic and plastidic activities of pyruvate kinase were assayed based on different pH optima reported before for purified Brassica napus pyruvate kinase isoforms (Plaxton et al, 2002 Arch Biochem Biophys 400: 54-62). Accordingly, the cytosolic enzyme has maximum activity at pH 6.7 while the plastidic isoform is inactive. At pH 8 the plastidic PK has maximum activity while the cytosolic PK activity has 60% of it's activity at pH 6.7. After measuring PK activity at pH 8.0 and at pH 6.7, the plastidic and cytosolic activities were calculated accordingly.

### Histological Procedures

Histochemical techniques applied to seeds as well as electron microscopy were done as described earlier (Borisjuk et al, 2005 New Phytologist 167: 761-776).

### Network Analysis and Visualization

The VANTED-Software (Rohn et al, 2012) was used for data analysis and visualisation. The elements were manually separated into four groups (metabolomics, enzyme activity, flux vector values, biomass), and a Pearson-correlation with these biomass components as target was performed. The correlation coefficient (r) was set to abs(r)=0.66. We then removed all the elements with smaller values, and colored elements in either red (positive correlation) or blue (negative correlation) with r-dependent alpha value. To further show the elements with highest correlation to each other, including the target, we performed a N:N Pearson-correlation with an absolute correlation value of abs(r)>=0.9. The correlation coefficients were visualised using edges. The strength of the correlation was visualised using edge thickness, whereas a correlation value of 1 is presented with thick edges and correlation value of 0.9 presented with very thin edges. Also the thickness is dependent by a quadratic factor. This was realized by first applying a linear transformation from the intervall of [0.9 . . . 1.0] to [0 . . . 1.0] with (y(r)=10*(r−0.9)) and then taking the root square of the target maximum width. Again the same color code as in the 1:n correlation was used.

### EXAMPLE 2

### Construction of T-DNA Vectors and Isolation of Transgenic Plants Overexpressing PEP Insensitive PPi-PFK and WR1

Using standard recombinant DNA techniques the following chimeric genes are created by operably linking the following DNA fragments:

### Recombinant PPi-PFK Encoding Gene

an oleosin promoter region (SEQ ID No. 7)
a DNA region encoding the amino acid sequence of SEQ ID No. 4 (PPi-PFK protein from Amycolatopsis methanolica.)
a transcription termination and polyadenylation signal from 3′ nopalinesynthase gene.

### Recombinant WRI1 Encoding Gene

an oleosin promoter region (SEQ ID No. 7)
a DNA region encoding WRI1 (SEQ ID No 6) from oil palm, codon optimized for expression in dicotyledonous plants (SEQ ID No. 5)
a transcription termination and polyadenylation signal from 3′ nopalinesynthase gene.

The chimeric genes are introduced between left and right T-DNA borders together with a selectable marker gene.

The T-DNA vector is introduced into an Agrobacterium strain comprising a helper Ti-plasmid using conventional methods. Hypocotyl explants of Brassica napus are obtained, cultured and transformed essentially as described by De Block et al. (1989), Plant Physiol. 91: 694) to transfer the chimeric genes into Brassica napus plants.

Trangenic Brassica napus plant lines are identified and analyzed for increased oil content.

1. A method to increase oil content in seeds or embryos of a Brassica plant, comprising the step of preventing feedback inhibition by phosphoenolpyruvate (PEP) of a pyrophosphate-dependent phosphofructokinase (PPi-PFK) present in cells of said seeds or said embryos.

2. The method according to claim 1, wherein said prevention of feedback inhibition is achieved by providing the plant cell with a PPi-PFK variant which is less sensitive to said feedback inhibition than a PPi-PFK endogenous to said plant.

3. The method according to claim 2, wherein said PPi-PFK variant is encoded by a variant allele in said plant cell.

4. The method according to claim 2, wherein said PPi-PFK variant is encoded by a transgene comprised within said cells.

5. The method according to claim 4, wherein said PPi-PFK variant is from an organism selected from the group of algae, bacteria, protozoa or archea.

6. The method according to claim 4, wherein said PPi-PFK variant is from an organism selected from the group of Thermoproteus tenax, Naegleria fowleri, Methylococcus capsulatus or Amycolatopsis methanolica.

7. The method according to claim 6, wherein said PPi-PFK variant is from Amycolatopsis methanolica.

8. The method according to claim 6, wherein said PPi-PFK variant comprises an amino acid sequence of any one of SEQ ID Nos: 1-4.

9. The method of claim 4, wherein said cells is provided with a DNA molecule comprising the following operably linked DNA fragments:
(a) a plant expressible promoter, preferably a seed-specific promoter;
(b) a DNA region encoding a PPi-PFK variant which is less sensitive to said feedback inhibition than a PPi-PFK endogenous to said Brassica plant, preferably a DNA region encoding a PPi-PFK comprising an amino acid sequence selected from the amino acid sequences of SEQ ID Nos. 1, 2, 3 or 4, preferably a heterologous DNA region; or a DNA region encoding a protein 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity with an amino acid sequence selected from the amino acid sequence of SEQ ID Nos. 1,2, 3 or 4, and having PPi-PFK enzymatic activity; and optionally
(c) a transcription termination and/or polyadenylation region functional in plant cells.

10. The method according to claim 9, wherein said DNA region comprises a nucleotide sequence which is codon-optimized to codon usage in plants, preferably dicotyledonous plants, preferably Brassica plants.

11. The method of claim 4 comprising the further step of providing said cells with one or more further recombinant DNA molecules comprising the following operably linked DNA fragments:
(a) a plant expressible promoter, preferably a seed-specific promoter;
(b) a DNA region, preferably a heterologous DNA region encoding a polypeptide selected from the following group:
(i) sucrose transporter capable of influencing sugar transport into the seed or embryo, such as AtSUC5;
(ii) Na-pyruvate/sodium:proton antiporter;
(iii) homeric acetyl-CoA carboxylase;
(iv) glycerol-3-phosphate dehydrogenase;
(v) transcription factor wrinkled 1;
(vi) AtABCA9 transporter;
(vii) Sn-2 acyltransferase
(viii) lysophosphatidic acid acyl transferase
(ix) glycerol-3-phosphate acyltransferase
(x) diacylglycerol acyltransferase; or
(xi) oleosin; and optionally
(i) a transcription termination and/or polyadenylation region functional in plant cells.

12. The method of claim 11, wherein said further recombinant DNA molecule encodes a transcription factor wrinkled 1, preferably comprising the amino acid sequence of SEQ ID No: 6 or a polypeptide having at least 90%, 91%, 92%, 93%, 94%, 95%, 96%, 97%, 98% or 99% sequence identity with said amino acid sequence.

13. The method of claim 4, comprising the further step of providing said cells with a further recombinant DNA molecule comprising the following operably linked DNA fragments:
(a) a plant expressible promoter, preferably a seed-specific promoter;
(b) a DNA region which encodes an inhibitory RNA capable of suppressing expression of sugar-dependent) triacylglycerol lipase.

14. The method according to claim 1, wherein said prevention of feedback inhibition is achieved by reducing the steady state level of PEP in said plant cells.

15. The method according to claim 13, wherein said reduction of the steady state level of PEP in said plant cells is achieved by increasing the level or activity of PEP carboxylase and/or PEP carboxykinase.

16. The method according to claim 15, wherein said level or activity PEP carboxylase and/or PEPcarboxykinase is increased by introduction into said plant cell of a recombinant DNA molecule comprising the following operably linked DNA fragments:
i. a plant expressible promoter, preferably a seed specific promoter
ii. a DNA region encoding a PEP carboxylase or PEP carboxykinase; and optionally
iii. a transcription termination and/or polyadenylation region functional in plant cells.

17. The method according to claim 1, wherein said plant is Brassica napus, Brassica campestris (rapa), Brassica juncea or Brassica carinata.

18. A Brassica plant, or seeds thereof, comprising in cells of it seeds or embryos, a pyrophosphate-dependent phosphofructokinase which is less sensitive to said feedback inhibition than a PPi-PFK endogenous to said plant.

19. The Brassica plant, or seeds thereof according to claim 18, wherein said less sensitive PPi-PFK is encoded by a transgene comprised within cells of said plant.

20. The Brassica plant, or seeds thereof according to claim 18, wherein said less sensitive PPi-PFK is from an organism selected from the group of algae, bacteria, protozoa or archea.

21. The Brassica plant, or seeds thereof according to claim 18, wherein said less sensitive PPi-PFK is from an organism selected from the group of Thermoproteus tenax, Naegleria fowleri, Methylococcus capsulatus or Amycolatopsis methanolica.

22. The Brassica plant, or seeds thereof according to claim 18, wherein said less sensitive PPi-PFK is from Amycolatopsis methanolica.

23. The Brassica plant, or seeds thereof according to claim 18, wherein said less sensitive PPi-PFK comprises an amino acid sequence of any one of SEQ ID Nos: 1-4.

24. The Brassica plant or seed thereof, according to claim 18 comprising a DNA molecule comprising the following operably linked DNA fragments:
(a) a plant expressible promoter, preferably a seed-specific promoter;
(b) a DNA region encoding a PPi-PFK which is less sensitive to said feedback inhibition than a PPi-PFK endogenous to said Brassica plant, preferably a DNA region encoding a PPi-PFK comprising an amino acid sequence selected from the amino acid sequences of SEQ ID Nos. 1, 2, 3 or 4, preferably a heterologous DNA region; or a DNA region encoding a protein 70%, 71%, 72%,73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity with an amino acid sequence selected from the amino acid sequence of SEQ ID Nos. 1,2, 3 or 4, and having PPi-PFK enzymatic activity; and optionally
(c) a transcription termination and/or polyadenylation region functional in plant cells.

25. The Brassica plant, or seeds thereof according to claim 18 comprising one or more further recombinant DNA molecules comprising the following operably linked DNA fragments:
(a) a plant expressible promoter, preferably a seed-specific promoter;
(b) a DNA region, preferably a heterologous DNA region encoding a polypeptide selected from the following group:
(i) sucrose transporter capable of influencing sugar transport into the seed or embryo, such as AtSUC5;
(ii) Na-pyruvate/sodium:proton antiporter;
(iii) homeric acetyl-CoA carboxylase;
(iv) glycerol-3-phosphate dehydrogenase;
(v) transcription factor wrinkled 1;
(vi) AtABCA9 transporter;
(vii) Sn-2 acyltransferase
(viii) lysophosphatidic acid acyl transferase
(ix) glycerol-3-phosphate acyltransferase
(x) diacylglycerol acyltransferase; or
(xi) oleosin; and optionally
(c) a transcription termination and/or polyadenylation region functional in plant cells.

26. The Brassica plant, or seeds thereof according to claim 25, wherein said further recombinant DNA molecule encodes a transcription factor wrinkled 1, preferably comprising the amino acid sequence of SEQ ID No: 6 or a polypeptide having at least 90%, 91%, 92%, 93%, 94%, 95%, 96%, 97%, 98% or 99% sequence identity with said amino sequence.

27. The Brassica plant, or seeds thereof according to claim 18 comprising a further recombinant DNA molecule comprising the following operably linked DNA fragments:
(a) a plant expressible promoter, preferably a seed-specific promoter;
(b) a DNA region which encodes an inhibitory RNA capable of suppressing expression of sugar-dependent 1 triacylglyccrol lipase.

28. A Brassica plant, or seeds thereof comprising a recombinant DNA molecule comprising the following operably linked DNA fragments:
i. a plant expressible promoter, preferably a seed specific promoter
ii. a DNA region encoding a PEP carboxylase or PEP carboxykinase ; and optionally
iii. a transcription termination and/or polyadenylation region functional in plant cells.

29. Cells, tissues, oil storage tissue, embryos or seeds of a plant according to claim 18 comprising a pyrophosphate-dependent phosphofructokinase which is less sensitive to said feedback inhibition than a PPi-PFK endogenous to said plant and/or comprising a recombinant PEP carboxylase or PEP carboxykinase and optionally further genetic modifications.

30. Oil derived from a plant according to claim 18.

31. A chimeric DNA comprising the following operably linked DNA fragments
a. a plant expressible promoter, preferably a seed-specific promoter;
b. a DNA region encoding a PPi-PFK which is less sensitive to said feedback inhibition than a PPi-PFK endogenous to said Brassica plant, preferably a DNA region encoding a PPi-PFK comprising an amino acid sequence selected from the amino acid sequences of SEQ ID Nos. 1, 2, 3 or 4, preferably a heterologous DNA region; or a DNA region encoding a protein 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity with an amino acid sequence selected from the amino acid sequence of SEQ ID Nos. 1,2, 3 or 4, and having PPi-PFK enzymatic activity; and optionally
c. a transcription termination and/or polyadenylation region functional in plant cells.

32. Use of a PPi-PFK enzyme which is less sensitive to feedback inhibition by PEP than a PPi-PFK endogenous to a Brassica plant to increase the oil content in seeds and or embryos of a Brassica plant.

33. A method to isolate variants of PPi-PFK enzyme which are less sensitive to feedback inhibition by PEP than a PPi-PFK endogenous to a Brassica plant comprising the steps of
a. generating a multitude of variant PPi-PFK enzymes from a PEP feedback inhibition sensitive PPi-PFK from a Brassica plant;
b. identifying the enzymatic activity of each of said variant PPi-PFK enzymes in the presence of PEP;
c. isolating those enzyme variants which have a greater enzymatic activity in the presence of PEP than the enzymatic activity of said PEP feedback inhibition sensitive PPi-PFK.

34. A method to increase oil content in cells of a plant comprising the steps of
a. isolating a variant of PPi-PFK which is less sensitive to feedback inhibition by PEP according to the method of claim 33;
b. introducing said variant of PPi-PFK in a Brassica plant, preferably by transcription from a DNA construct encoding said PPi-PFK.

35. A method to isolate a plant cell or plant comprising a variant allele encoding a PPi-PFK variant enzyme which is less sensitive to feedback inhibition by PEP comprising the steps of
a. providing a population of plant cells or plants, each comprising a multitude of variant PPi-PFK;
b. identifying the enzymatic activity of each of said PPi-PFK enzymes in the presence of PEP;
c. isolating those plant cells or plants comprising enzyme variants which have a greater enzymatic activity in the presence of PEP than the enzymatic activity of said feedback inhibition sensitive PPi-PFK.

36. A plant cell or plant obtained by the method of claim 35.

37. A method of producing food, feed, or an industrial product comprising
a. obtaining the plant or a part thereof or a seed thereof, of claim 18; and
b. preparing the food, feed or industrial product from the plant or part thereof

38. The method of claim 37 wherein
a. the food or feed is oil, meal, grain, starch, flour or protein; or
b. the industrial product is biofuel, fiber, industrial chemicals, a pharmaceutical or a nutraceutical.
